(19) United States
(12) Patent Application Publication
(10) Pub. No.: US 2010/0204477 A1
(43) Pub. Date: Aug. 12, 2010

(54) Methods and Processes For Syntheses and Manufacture of Antimicrobial 1(Ortho-Fluorophenyl)dihydropyridones

(76) Inventors: Qiang Wang, (US); Feng Zhou, (US); Jinqian Liu, Palo Alto, CA (US); Mikhail Fedorovich Gordeev, Castro Valley, CA (US)

Correspondence Address: JONES DAY, 222 EAST 41ST ST, NEW YORK, NY, 10017, US

(21) Appl. No.: 12/701,298

(22) Filed: Feb. 5, 2010

Related U.S. Application Data
(60) Provisional application No. 61/150,553, filed on Feb. 6, 2009.

(30) Foreign Application Priority Data
Feb. 6, 2009 (CN) 200910046002.3

(51) Int. Cl. C07D 413/14; C07D 413/10

(52) U.S. Cl. 546/271.4

(57) ABSTRACT

Provided herein are methods and processes for synthesis and manufacture of compounds of formula I:

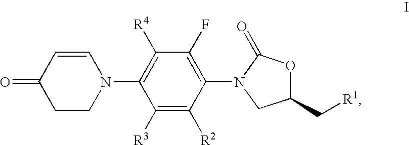

or its crystal forms, pharmaceutical acceptable salts, prodrugs, hydrates, or solvates thereof.

### CROSS REFERENCE TO RELATED APPLICATIONS

The present application claims the benefit under 35 U.S.C. §119 of priority of U.S. Provisional Application No. 61/150,553, filed Feb. 6, 2009, and of Chinese Patent Application No. 200910046002.3, filed Feb. 6, 2009, the contents of each of which are hereby incorporated by reference in their entireties.

### FIELD OF THE INVENTION

Provided herein are methods and processes for synthesis and manufacture of novel 1-(ortho-fluorophenyl)dihydropyridone derivatives of oxazolidinones. Said compounds have potent activities against widespread pathogenic bacteria and are useful for treatment of infections in mammals.

### BACKGROUND OF THE INVENTION

Due to an increasing antibiotic resistance, novel classes of antibacterial compounds with a new mode of action are acutely needed for the treatment of bacterial infections.

Among newer antibacterial agents, oxazolidinone compounds are the most recent synthetic class of antimicrobials active against a number of pathogenic microorganisms. To date, a sole antibacterial of this class linezolid (Zyvox®) has been approved for a treatment of select gram-positive infections. While oxazolidinones represented by this drug are useful for the treatment of microbial infections, their utility is limited due to modest antibacterial potency and serious adverse effects. Among these, monoamine oxidase inhibition and myelosuppression or bone marrow toxicity are key factors limiting linezolid utility, as reflected in warnings in the drug's prescribing information for Zyvox®. Thus, introduction and manufacture of newer agents of this class with and improved potency and safety profile is urgently needed to combat life-threatening infections in human and animals.

### SUMMARY OF THE INVENTION

Provided herein are methods and processes for synthesis and manufacture of novel pharmaceutical 1-(ortho-fluoroaryl)dihydropyridone oxazolidinone compounds with useful antibacterial activity. The term ortho-fluorophenyl indicates the presence of the mandatory F (fluorine) substituent in a position 2 of a respective aryl (e.g., phenyl) oxazolidinone, i.e., F at the aryl (e.g., phenyl) group site adjacent to the oxazolidinone ring nitrogen.

Compounds prepared by the methods and processes disclosed herein, including those disclosed in U.S. Patent Publication No. US2009/0048305 A1 and International Patent Publication No. WO 2009/02061 A1 (the contents of each of which are incorporated herein by reference in their entireties), can combine high antibacterial activity with reduced monoamine oxidase inhibition. Furthermore, ortho-fluorophenyl oxazolidinones disclosed herein can offer a beneficially reduced myelosuppression. The compounds are useful as antibacterial agents for treatment of infections including, but not limited to, skin infections, soft tissue infections, bacteremia, respiratory tract infections, urinary tract infections, bone infections, and eye infections.

Provided herein are provides methods and processes for synthesis and manufacture of compounds of formula I, or its crystal forms, pharmaceutical acceptable salts, prodrugs, hydrates, or solvates thereof, as well as intermediates (including compounds of formulae II-IX below) useful for the preparation of said compounds of formula I:

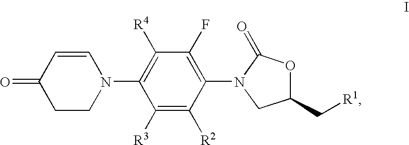

wherein

R¹ is NHC(═O)R⁵, OH, R⁵OH, NHC(═S)R⁵, NHC(═NCN)R⁵, NH-Het¹, O-Het¹, S-Het¹, or Het²; wherein R⁵ is H, NH₂, NHC₁₋₄alkyl, C₁₋₄alkyl, C₃₋₆cycloalkyl, C₂₋₄alkenyl, C₂₋₄alkynyl, C₁₋₄heteroalkyl, Het¹, Het², (CH₂)ₘC(═O)C₁₋₄alkyl, OC₁₋₄alkyl, SC₁₋₄alkyl, (CH₂)ₘC₃₋₆cycloalkyl, (CH₂)ₘC(═O)-aryl, or (CH₂)ₘC(═O)—Het¹; m is 0, 1, or 2; Het¹ is independently a C-linked 5 or 6 membered heterocyclic ring having 1 to 4 heteroatoms selected from the group consisting of oxygen, nitrogen, and sulfur within the ring; and Het² is independently an N-linked 5 or 6 membered heterocyclic ring having 1 to 4 nitrogen atoms and optionally having one oxygen or sulfur within the ring;

R² is H or F; and

R³ and R⁴ are independently H, F, Cl, CN, or OH.

In certain aspects, provided herein are methods and processes for synthesis and manufacturing of compounds of formula II comprising combining a 4-piperidone compound with a substituted 2-fluoronitrobenzene compound in an aprotic solvent and an optional base to form an N-aryl-4-piperidone compound of formula II:

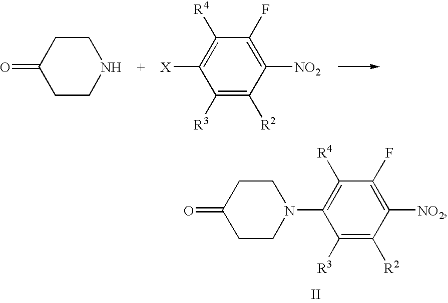

wherein X is F, Cl, Br, I.

In other aspects, provided herein are methods and processes for synthesis and manufacturing of compounds of formula III comprising combining an N-aryl-4-piperidone compound of formula II with a trialkylsilyl compound R³SiX (wherein X is halo, alkylsulfonate, or triflate; and wherein R is C₁₋₁₂alkyl, C₃₋₆cycloalkyl, aryl, or alike) and a base in an aprotic solvent to form a silyl enol ether compound of formula III:

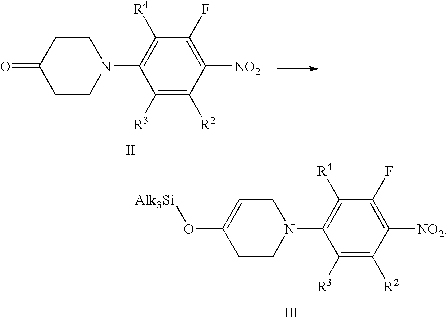

In additional aspects, provided herein are methods and processes for synthesis and manufacturing of compounds of formula IV comprising combining a silyl enol ether compound of formula III, O-alkyl-O′-allyl carbonate, a Pd(II) compound, and an optional fluorinated nitrobenzene compound in an aprotic solvent to form an N-aryl-4-(2,3-dihydro)pyridone compound of formula IV:

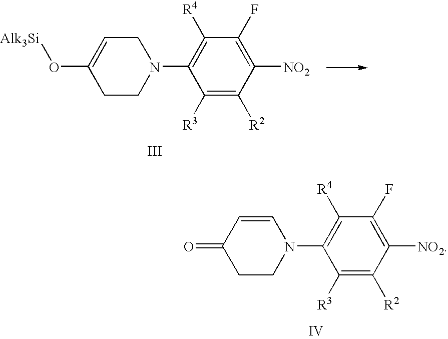

In other aspects, provided herein are methods and processes for synthesis and manufacturing of compounds of formula V comprising combining an N-aryl-4-(2,3-dihydro)pyridone compound of formula IV with a metal powder (selected from Fe, Sn, Ce, Ti, or Zn) in acidic aqueous solution, or optionally combining N-aryl-4-(2,3-dihydro)pyridone compound of formula IV with a hydrogen source and a Pd catalyst, to form an aniline of formula V:

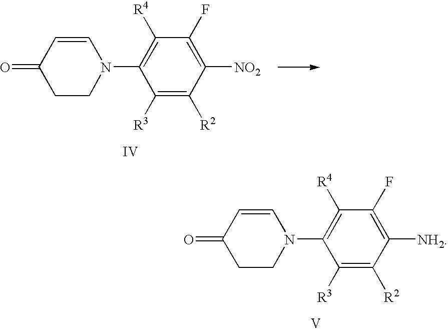

In additional aspects, provided herein are methods and processes for synthesis and manufacturing of compounds of formula VI comprising combining an aniline compound of formula V, alkyl chloroformate, and an optional base in aprotic solvent to form a carbamate compound of formula VI:

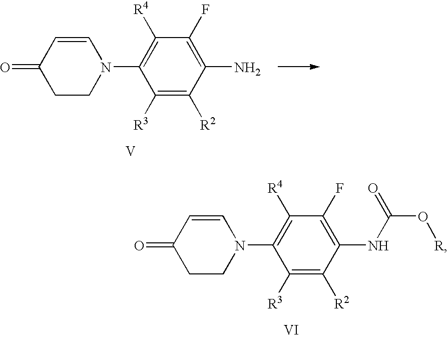

wherein R is C₁₋₁₂alkyl, C₃₋₆cycloalkyl, aryl, heteroaryl, or arylalkyl.

In other aspects, provided herein are methods and processes for synthesis and manufacturing of compounds of formula VII comprising combining a carbamate compound of formula VI, an epoxide compound or a chlorohydrin compound, and a base in an aprotic solvent to form an oxazolidinone compound of formula VII:

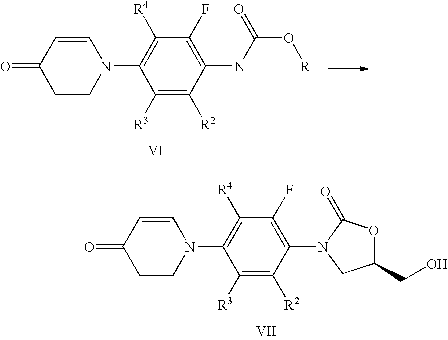

wherein R is C₁₋₁₂alkyl, C₃₋₆cycloalkyl, aryl, heteroaryl, or arylalkyl.

In further aspects, provided herein are methods for converting the compound of formula VII, optionally through one or more intermediates, into the compound of formula I, for R¹ other than OH, as disclosed herein:

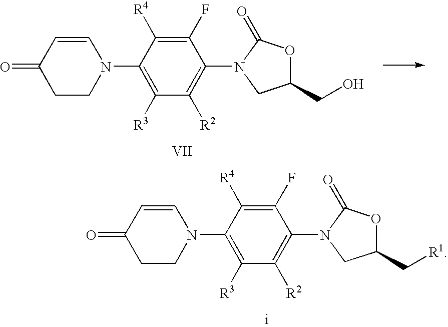

In other aspects, provided herein are methods and processes for synthesis and manufacturing of compounds of formula VIII comprising combining an oxazolidinone compound of formula VII and a compound R₉SO₂Cl in an aprotic solvent and an optional base to form a sulfonate oxazolidinone compound of formula VIII:

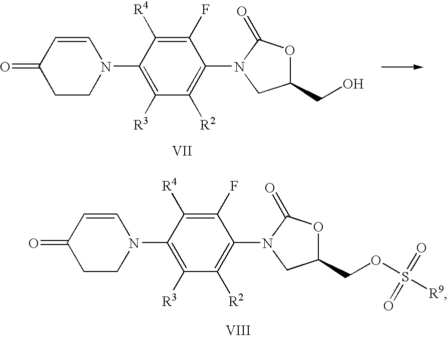

wherein R⁹ is C₁₋₄alkyl, trifluoromethyl, aryl, nitrophenyl, para-methylphenyl or alike group.

In additional aspects, provided herein are methods and processes for synthesis and manufacturing of compounds of formula IX comprising combining a sulfonate compound of formula VIII, a substituted heterocyclic compound of the formula 3-(PG)NH-5-R⁶-isoxazole, and an optional base in an aprotic solvent to form a compound of formula IX:

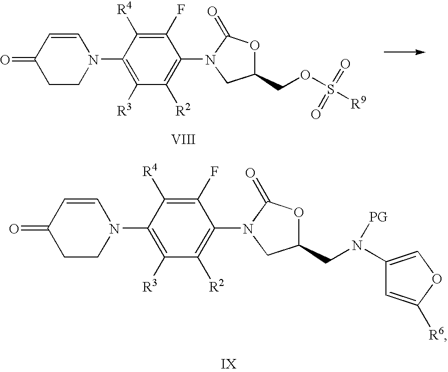

wherein PG is H or N-protective substituent selected from C₁₋₆alkoxycarbonyl, benzyloxycarbonyl, trichloroethoxycarbonyl, tert-butoxycarbonyl, para-methoxybenzyl, dimethoxybenzyl, or alike group.

In other aspects, provided herein are methods and processes for synthesis and manufacturing of compounds of formula IX comprising combining an alcohol compound of formula VII, a substituted heterocyclic compound of the formula 3-(PG)NH-5-R⁶-isoxazole, a trisubstituted phosphine, and an azodicarbonyl compound R′C(═O)—N═N—C(═O)R′ (wherein R¹ is C₁₋₆alkoxy, C₃₋₆cycloalkoxy, or C₁₋₆alkoxyamino group) in an aprotic solvent to form a compound of formula IX.

In additional aspects, provided herein are methods and processes for synthesis and manufacturing of compounds of formula I comprising combining a compound of formula IX and a N-protection removing agent to form a compound of formula I of the following structure:

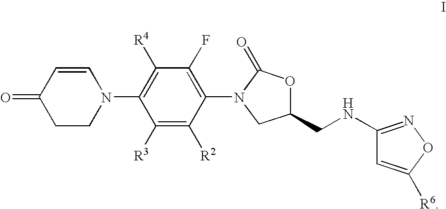

The independent alkyl, alkenyl, or cycloalkyl groups at each occurrence above are optionally substituted with one, two, or three substituents selected from the group consisting of halo, aryl, Het¹, and Het². Het¹ at each occurrence is independently a C-linked 5 or 6 membered heterocyclic ring having 1 to 4 heteroatoms selected from the group consisting of oxygen, nitrogen, and sulfur within the ring. Het² at each occurrence is independently a N-linked 5 or 6 membered heterocyclic ring having 1 to 4 nitrogen and optionally having one oxygen or sulfur within the ring.

In another aspect, provided herein are crystal forms of compounds of formula I, for example, polymorphs of anhydrous or solvated crystal forms of compounds of formula I.

### BRIEF DESCRIPTION OF THE FIGURES

FIG. 1 provides a different scanning calorimetry plot of the Form A crystal of the compound of Example 3.

FIG. 2 provides an X-ray powder diffraction plot of the Form A crystal of the compound of Example 3 (KBr pellet).

FIG. 3 provides an infrared spectrum of the Form A crystal of the compound of Example 3.

### DETAILED DESCRIPTION OF THE INVENTION

Unless stated otherwise, the following terms used in the specification and Claims have the meanings given below.

The carbon atom content of various hydrocarbon-containing moieties is indicated by a prefix designating the minimum and maximum number of carbon atoms in the moiety, i.e., the prefix indicates a moiety of the integer “i” to the integer “j” carbon atoms, inclusive. Thus, for example, C₁₋₇ alkyl refers to alkyl of one to seven carbon atoms, inclusive.

Group R^{#} is same as R_{#} or R#: R¹ is same as R₁ or R1, etc.

t-Alk is same as tert-Alk or tert-Alk: t-Bu is same as tert-Bu or tert-Bu.

TMS is trimethylsilyl, TMSOTf is trimethylsilyl triflate, and TMSHal is trimethylsilyl halide.

The terms “alkyl,” “alkenyl,” etc. refer to both straight and branched groups, but reference to an individual radical such as “propyl” embraces only the straight chain radical, a branched chain isomer such as “isopropyl” being specifically referred to. The alkyl, alkenyl, etc., group may be optionally substituted with one, two, or three substituents selected from the group consisting of halo, aryl, Het¹, or Het². Representative examples include, but are not limited to, difluoromethyl, 2-fluoroethyl, trifluoroethyl, —CH═CH-aryl, —CH═CH-Het¹, —CH₂-phenyl, 1-phenyl-1,1-di(tert-butyl)methyl, and the like.

The term “cycloalkyl” means a cyclic saturated monovalent hydrocarbon group of three to six carbon atoms, e.g., cyclopropyl, cyclohexyl, and the like. The cycloalkyl group may be optionally substituted with one, two, or three substituents selected from the group consisting of halo, aryl, Het¹, or Het².

The term “heteroalkyl” means an alkyl or cycloalkyl group, as defined above, having a substituent containing a heteroatom selected from N, O, or S(O)ₙ, where n is an integer from 0 to 2, including, hydroxy (OH), C₁₋₄alkoxy, amino, thio (—SH), and the like. Representative substituents include —NRₐR_{b}, —ORₐ, or —S(O)ₙR_{c}, wherein Rₐ is hydrogen, C₁₋₄alkyl, C₃₋₆cycloalkyl, optionally substituted aryl, optionally substituted heterocyclic, or —COR (where R is C₁₋₄alkyl); R_{b} is hydrogen, C₁₋₄alkyl, —SO₂R (where R is C₁₋₄alkyl or C₁₋₄hydroxyalkyl), —SO₂NRR′ (where R and R′ are independently of each other hydrogen or C₁₋₄alkyl), —CONR′R″ (where R′ and R″ are independently of each other hydrogen or C₁₋₄alkyl); n is an integer from 0 to 2; and R_{c} is hydrogen, C₁₋₄alkyl, C₃₋₆cycloalkyl, optionally substituted aryl, or NRₐR_{b} where Rₐ and R_{b} are as defined above. Representative examples include, but are not limited to, 2-methoxyethyl (—CH₂CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to phenyl, biphenyl, or naphthyl, optionally substituted with 1 to 3 substituents independently selected from halo, —C₁₋₄alkyl, —OH, —OC₁₋₄alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or —C₁₋₄alkyl. Likewise, the term phenyl refers to the phenyl group optionally substituted as above.

The term “heterocyclic ring” refers to an aromatic ring or a saturated or unsaturated ring that is not aromatic of 3 to 10 carbon atoms and 1 to 4 heteroatoms selected from the group consisting of oxygen, nitrogen, and S(O)ₙ within the ring, where n is defined above. The heterocyclic ring may be optionally substituted with halo, —C₁₋₄alkyl, —OH, —OC₁₋₄ alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or C₁₋₄alkyl.

Examples of heterocylic rings include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiadiazole tetrazole, thiazolidine, thiophene, benzo[b]thiophene, morpholinyl, thiomorpholinyl (also referred to as thiamorpholinyl), piperidinyl, pyrrolidine, tetrahydrofuranyl, 1,3-benzoxazine, 1,4-oxazine-3-one, 1,3-benzoxazine-4-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one and the like. Heterocyclic rings include unsubstituted and substituted rings.

Specifically, Het¹ (same as het¹, Het₁ or het₁) refers to a C-linked five- (5) or six- (6) membered heterocyclic ring, including bicyclic rings. Representative examples of “Het¹” include, but are not limited to, pyridine, thiophene, furan, pyrazole, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, 1,2,4-dithiazolone, or 3-azabicyclo[3.1.0]hexan-6-yl.

Het² (same as het², Het₂, or het₂) refers to an N-linked five- (5) or six- (6) membered heterocyclic ring having 1 to 4 nitrogen atoms, and optionally having one oxygen or sulfur atom, including bicyclic rings. Representative examples of “Het²” include, but are not limited to pyrrolyl, imidazolyl, pyrazolyl, 1,2,3-triazolyl, 1,2,4-triazolyl, 1,2,3,4-tetrazolyl, isoxazolidinonyl group, 3-azabicyclo[3.1.0]hexan-3-yl, 1,3,9,9a-tetrahydrooxazolo[3,4-a]indol-1-yl, 2-alkylpyrrolo[3,4-c]pyrazol-5(2H,4H,6H)-yl, and 5H-pyrrolo[3,4-b]pyridin-6(7H)-yl.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds disclosed herein may possess one or more asymmetric centers; such compounds can therefore be produced as individual (R)- or (S)-stereoisomers or as mixtures thereof. Unless indicated otherwise, the description or naming of a particular compound in the specification and Claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A hydrogen (H) or carbon (C) substitution for compounds of formula I include a substitution with any isotope of the respective atom. Thus, a hydrogen (H) substitution includes a ¹H, ²H (deuterium), or ³H (tritium) isotope substitution, as may be desired, for example, for a specific therapeutic or diagnostic therapy, or metabolic study application. Optionally, a compound provided herein may incorporate a known in the art radioactive isotope or radioisotope, such as ³H, ¹⁵O, ¹²C, or ¹³N isotope, to afford a respective radiolabeled compound of formula I.

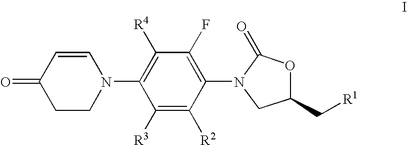

In certain aspects, R² in a compound of formula I is H, and R³ and R⁴ are both F.

In certain aspects, at least one substitutent R², R³, and R⁴ in a compound of formula I is F.

In certain aspects, R¹ in a compound of formula I is OH.

In certain aspects, R¹ in a compound of formula I is NH(C═O)OC₁₋₆alkyl.

In certain aspects, R¹ in a compound of formula I is NH(C═O)C₁₋₆alkyl.

In certain aspects, R′ in a compound of formula I is 4-R⁷-triazole-1-yl, wherein R⁷ is H, F, CN, or C₁₋₆alkyl.

In certain aspects, R′ in a compound of formula I is (5-R⁶-isoxazole-3-yl)oxy or (5-R⁶-isoxazole-3-yl)amino, wherein R⁶ is H or C₁₋₆alkyl.

In certain aspects, R¹ is (isoxazole-3-yl)amino, R² is H, and R³ and R⁴ are both F.

In certain aspects, provided herein are methods and processes for synthesis and manufacture of a compound of formula I of any the following structures:

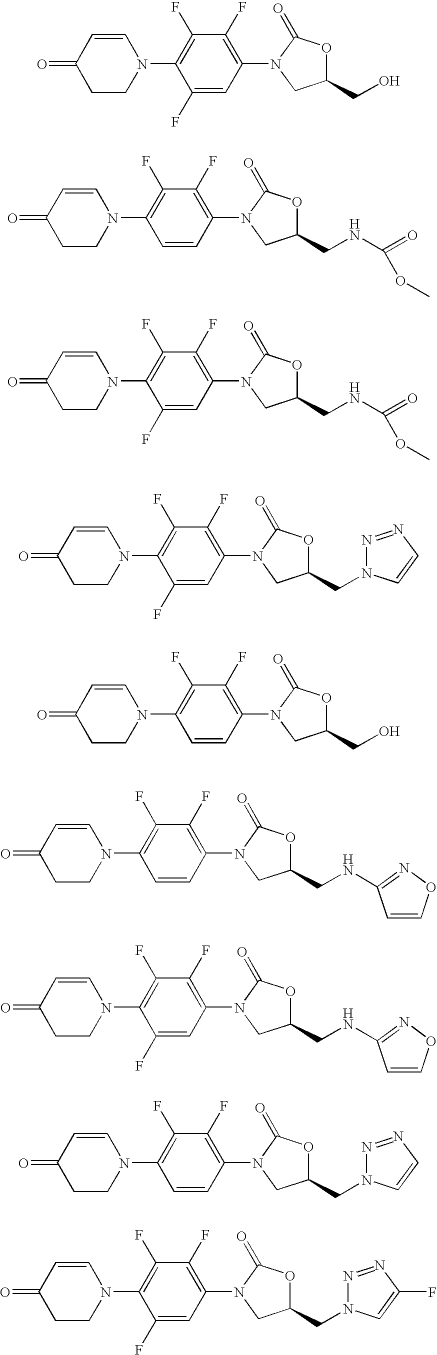

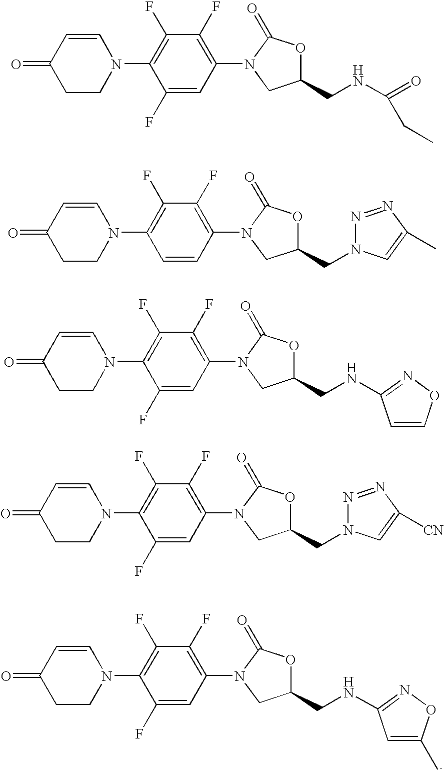

In certain aspects, aforementioned methods and processes comprise one or more of the following steps (a-g) below:

a) combining a 4-piperidone compound with a substituted 2-fluoronitrobenzene compound in an aprotic solvent and an optional base to form an N-aryl-4-piperidone compound of formula II:

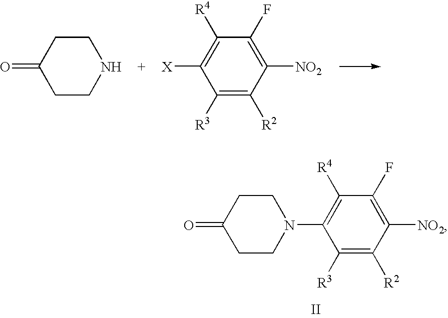

wherein X is F, Cl, Br, I;

b) combining an N-aryl-4-piperidone compound of formula II with a trialkylsilyl compound Alk₃SiX (wherein X is halo, alkylsulfonate, or triflate) and a base in an aprotic solvent to form a silyl enol ether compound of formula III

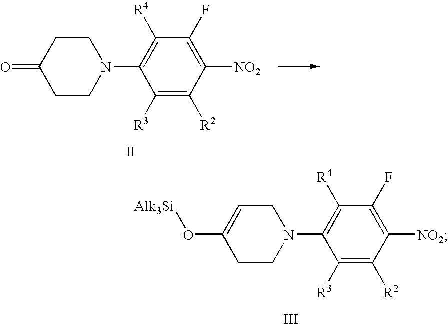

c) combining a silyl enol ether compound of formula III, O-alkyl-O′-allyl carbonate, a Pd(II) compound, and a fluorinated nitrobenzene compound in an aprotic solvent to form an N-aryl-4-(2,3-dihydro)pyridone compound (also referred to as 1-aryldihydropyridone compound) of formula IV:

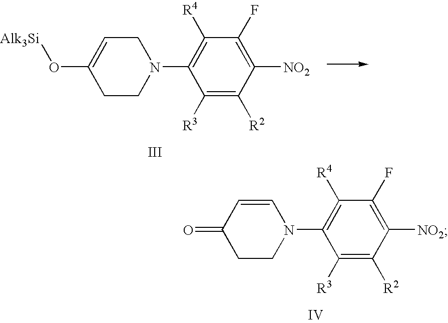

d) combining an N-aryl-4-(2,3-dihydro)pyridone compound of formula IV with a metal powder (selected from Fe, Sn, Ce, Ti, or Zn) in acidic aqueous or acidic organic solution, or combining N-aryl-4-(2,3-dihydro)pyridone compound of formula IV with a hydrogen source and a Pd, Pt, Fe, or Ni catalyst, to form an aniline of formula V:

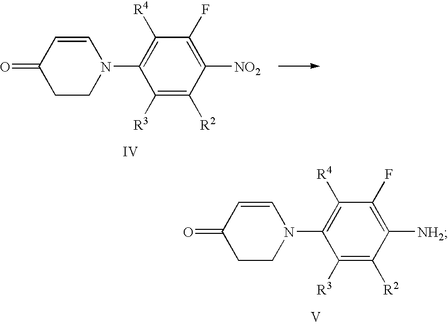

e) combining an aniline compound of formula V, alkyl chloroformate, and a base in aprotic solvent to form a carbamate compound of formula VI:

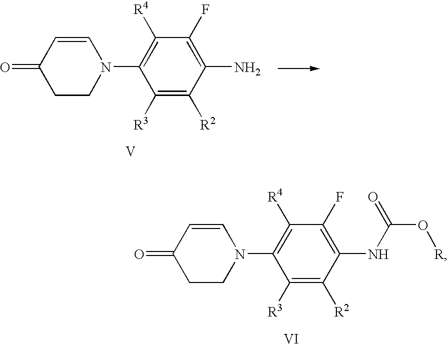

wherein R is C₁₋₁₂alkyl, C₃₋₆cycloalkyl, aryl, heteroaryl, or arylalkyl;

f) combining a carbamate compound of formula VI and an epoxide compound, or a chlorohydrin compound and a base in an aprotic solvent, to form an oxazolidinone compound of formula VII:

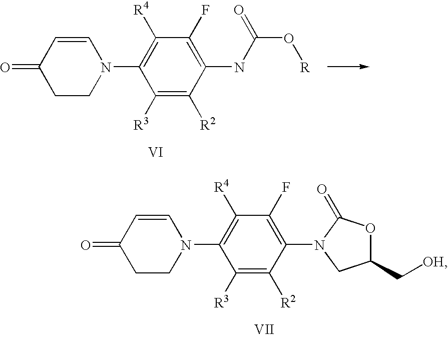

wherein R is C₁₋₁₂alkyl, C₃₋₆cycloalkyl, aryl, heteroaryl, or arylalkyl;

In certain aspects, aforementioned methods and processes comprise any of the following steps (g-i):

g) combining an oxazolidinone compound of formula VII and a compound R⁹SO₂Cl in an aprotic solvent and an optional base to form a sulfonate oxazolidinone compound of formula VIII:

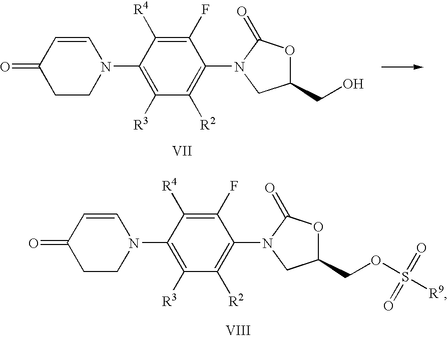

wherein R₉ is C₁₋₁₂alkyl, trifluoromethyl, aryl, nitrophenyl, para-methylphenyl, heteroaryl;

h) combining a sulfonate compound of formula VIII, a substituted heterocyclic compound of the formula 3-(PG)NH-5-R⁶-isoxazole, and an optional base in an aprotic solvent to form a compound of formula IX:

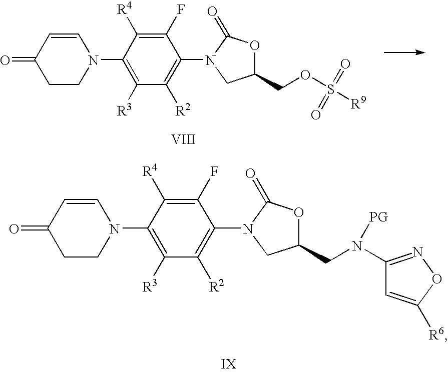

wherein PG is H or N-protective substituent selected from C₁₋₆alkoxycarbonyl, benzyloxycarbonyl, trichloroethoxycarbonyl, tert-butoxycarbonyl, para-methoxybenzyl, dimethoxybenzyl, or alike group;

i) combining a compound of formula IX and a N-protection removing agent to form a compound of formula

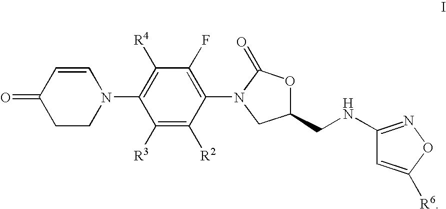

In certain aspects, a substituted 2-fluoronitrobenzene is 2,3,4-trifluoronitrobenzene, or 2-,4-difluoronitrobenzene, or 2-fluoro-1,3-dinitrobenzene, or 2,3,4,5-tetrafluoronitrobenzene; the aprotic solvent is NMP (N-methylpyrrolidin-2-one), DMF (N,N-dimethylformamide), DMA (N,N-dimethylacetamide), or dioxane; the base is N,N-diisopropyl-N′-ethylamine, triethylamine, DBU (1,8-diazabicyclo[5.4.0]undec-7-ene), or pyridine; and the process is performed at temperatures between −20 and 60° C.

In certain aspects, an Alk₃SiX reagent used to produce a compound of formula III is TMSCl, TMSBr, or TMSOTf; the aprotic solvent is tetrahydrofuran; the base is triethylamine; and the process is performed at temperatures between −10 and 50° C.

In certain aspects, O-alkyl-O′-allyl carbonate used to produce a compound of formula IV is O-methyl-O′-allyl carbonate, diallyl carbonate, or AllylO—C(═O)—OCH₂CH₂O—C(═O)—OAllyl, or alike reagent; the Pd(II) compound is Pd(OAc)₂; the aprotic solvent is DMSO, NMP, DMF, or MeCN; and the process is performed at temperatures between 0 and 60° C.

In certain aspects, the metal employed to produce a compound of formula V is iron; the hydrogen source is H₂ gas, or organic hydrogen source such as cyclohexene or formic acid reagent; the Pd catalyst is Pd/C, or Pd/CaCO₃, Pd(OH)₂, or Pd/C/quinoline.

In certain aspects, an alkyl chloroformate reagent used to produce a compound of formula VI is isobutyl chloroformate, or benzyl chloroformate; the solvent is DCM; the base is pyridine; and the process is performed at temperatures between −10 and 60° C.

In certain aspects, the epoxide reagent used to produce a compound of formula VII is (R)-glycidyl butyrate; the solvent is THF or MeCN, or a mixture of THF and MeCN in any ratio; the base is lithium or potassium t-butoxide; and the process is performed at temperatures between −20 and 60° C.

In other aspects, the epoxide reagent used to produce a compound of formula VII is (R)-glycidol.

In certain aspects, a base reagent used in production of a compound of formula VI is lithium or potassium t-butoxide, potassium tert-amylate, KOTMS, or sodium isopropoxide, or alike reagent; and the process is performed at temperatures between −10 and 25° C.

In certain aspects, the chlorohydrin reagent used to produce a compound of formula VII is epichlorohydrin; the solvent is THF or MeCN, or a mixture of THF and MeCN in any ratio; the base is lithium or potassium t-butoxide; and the process is performed at temperatures between −20 and 60° C.

In certain aspects, a R⁹SO₂Cl reagent used to produce a compound of formula VIII is CH₃SO₂Cl; the base is triethylamine.

In certain aspects, a substituted aminoheterocycle used to produce a compound of formula IX is 3-[N-(tert-butoxycarbonyl)amino]isoxazole; the aprotic solvent is DMF; and the base is potassium t-butoxide.

In certain aspects, a base reagent used to produce a compound of formula IX is potassium t-butoxide.

In certain aspects, the N-protection removing agent used to produce compound of formula I is 10-38% aqueous HCl or TMSCl.

In certain aspects, the N-protection removing agent used to produce compound of formula I is 25-38% aqueous HCl in EtOH/EtOAc, wherein the HCl, EtOH and EtOAc are in any ratio.

In certain aspects, the N-protection removing agent used to produce compound of formula I is 38% aqueous HCl in EtOH/EtOAc, wherein the HCl, EtOH and EtOAc are in any ratio between 1:1:1 to 3:1:3, respectively.

In certain aspects, the fluorinated nitrobenzene compound used in the production of a compound formula IV is 2,3,4,5-tetrafluoronitrobenzene or 2,3,4-trifluoronitrobenzene, employed in amounts in the range of 5-70 molar %.

In certain aspects, the fluorinated nitrobenzene compound used in the production of a compound formula IV is 2,3,4,5-tetrafluoronitrobenzene, or 2,3,4-trifluoronitrobenzene, in an amount of 40-60 molar %.

In another aspect, provided herein are anhydrous or solvated crystal forms of a compound according to formula I:

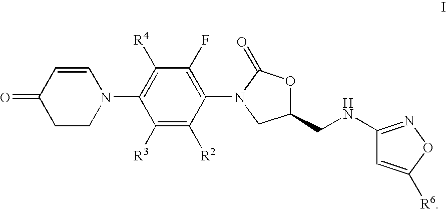

In certain embodiments, the crystal forms can be prepared by crystallization of the following compound of formula I:

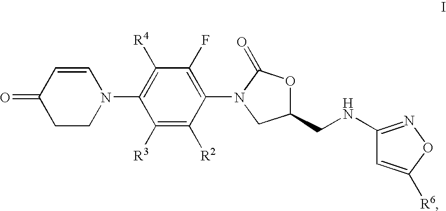

from a system containing one or more solvents, such as, but not limited to ethanol, ethyl acetate, hexane, petroleum ether, methyl t-butyl ether, and water.

In certain aspects, provided herein is a Form A crystal of the following compound of Example 3:

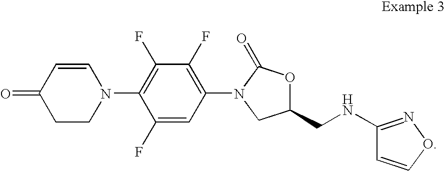

In certain embodiments, the Form A crystal of the compound of Example 3 is anhydrous.

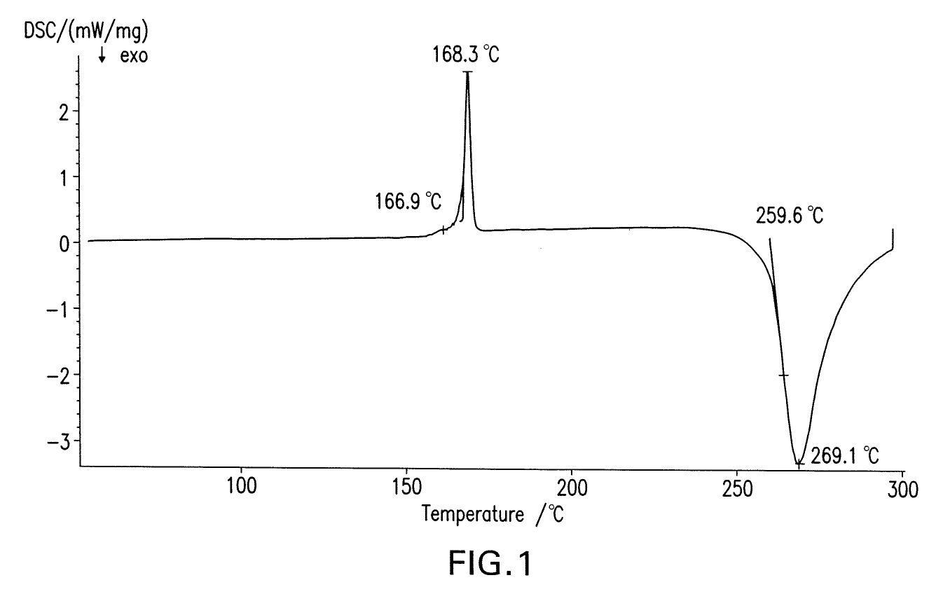

In certain embodiments, the Form A crystal of the compound of Example 3 has a differential scanning calorimetry pattern similar to that of FIG. 1. In certain embodiments, when examined by differential scanning calorimetry, the Form A crystal of the compound of Example 3 shows a single endothermic event, consistent with a crystal melting process. In certain embodiments, when examined by differential scanning calorimetry, the Form A crystal of the compound of Example 3 shows a single endothermic event at about 166 to about 168° C., consistent with a crystal melting process.

In certain embodiments, the Form A crystal of the compound of Example 3 has a melting temperature of between about 166.9 to about 168.3° C.

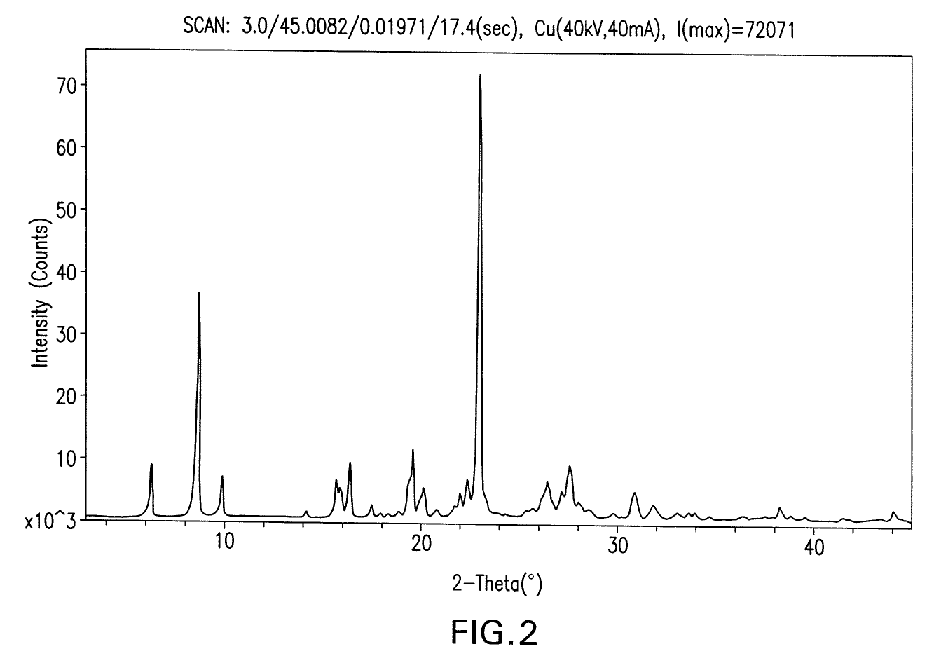

In certain embodiments, the Form A crystal of the compound of Example 3 has an X-ray powder diffraction pattern similar to that of FIG. 2 using Cu Kα radiation (e.g. 1.5406 Angstrom, 40 kV, 40 mA). In certain embodiments, the Form A crystal of the compound of Example 3 has an X-ray powder diffraction pattern with major peaks at about 8.5 to about 8.6, and at about 23.0 to 23.1 °2θ using Cu Kα radiation. In certain embodiments, the Form A crystal of the compound of Example 3 form has X-ray powder diffraction pattern peaks at one or two of the following approximate positions: about 8.5 to about 8.6, and about 23.0, using Cu Kα radiation.

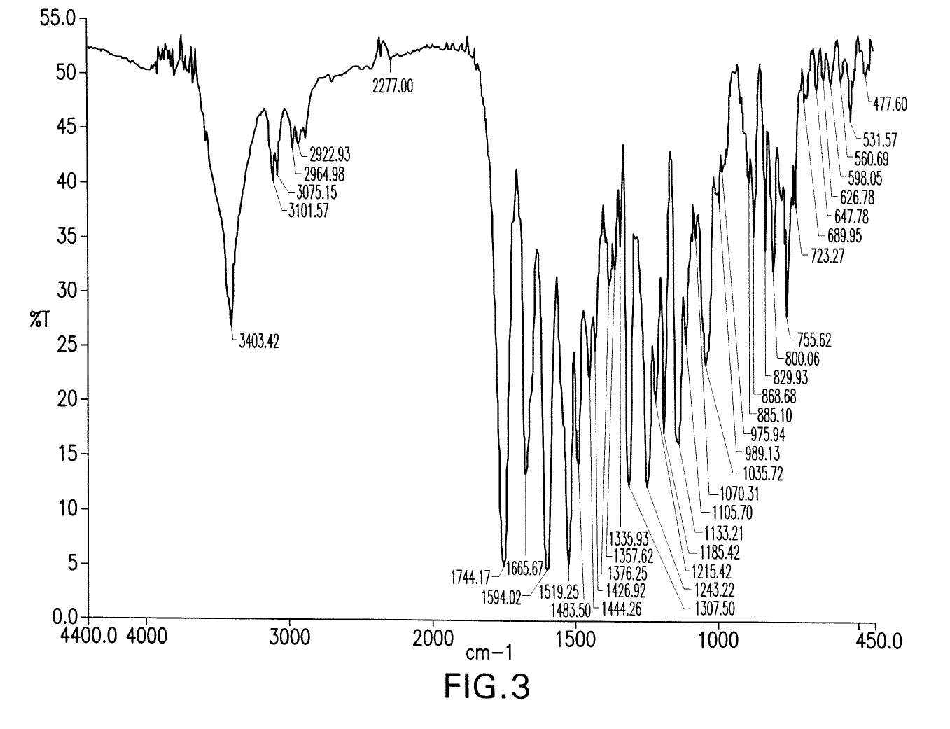

In certain embodiments, the Form A crystal of the compound of Example 3 has an infrared spectrum similar to that depicted in FIG. 3. In certain embodiments, the Form A crystal of the compound of Example 3 has infrared peaks at one, two, three, four, five, or more of the positions indicated in FIG. 3. In particular embodiments, the Form A crystal of the compound of Example 3 has one, two, three, four, or five infrared peaks at the following approximate positions: about 3403.4, about 1744.2, about 1665.7, about 1594.0, and about 1519.3 cm⁻¹.

In certain embodiments, the Form A crystal of the compound of Example 3 has an ultraviolet spectrum with a maximum absorption peak at about 318 nm.

The Form A crystal of the compound of Example 3 can be made by any method apparent to those of skill in the art based upon the teachings disclosed herein.

In certain embodiments, the Form A crystal can be prepared by crystallization of the following compound of Example 3:

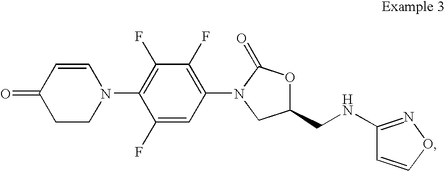

from a system containing one or more solvents, such as, but not limited to ethanol, ethyl acetate, hexane, petroleum ether, methyl t-butyl ether, and water.

The term “mammal” refers to all mammals including humans, livestock, and companion animals.

“Salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

(2) salts farmed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Prodrug” means any compound which releases in vivo an active parent drug of a compound disclosed herein when such prodrug is administered to a mammalian subject. Various prodrugs have been described, for example, in the following publications: Alexander et al. J. Med. Chem. 1988, p. 318; Alexander et al. J. Med. Chem., 1991, p. 78; Murdock et al. J. Med. Chem., 1993, p. 2098; Davidsen et al. J. Med. Chem., 1994, p. 4423; Robinson et al. J. Med. Chem., 1996, p. 10; Keyes et al. J. Med. Chem., 1996, p. 508; Krise et al. J. Med. Chem., 1999, p. 3094; Rahmathullah et al. J. Med. Chem., 1999, p. 3994; Zhu et al. Bioorg. Med. Chem. Lett., 2000, p. 1121; Sun et al., J. Med. Chem., 2001, p. 2671; Ochwada et al., Bioorg. Med. Chem. Lett., 2003, p. 191; Sohma et al. Med. Chem., 2003, p. 4124; Ettmayer et al. J. Med. Chem., 2004, p. 2393; Stella et al., Adv. Drug Delivery Rev., 2007, p. 677, Josyula et al. International Patent Publication No. WO 2005/028473; Rhee et al. International Patent Publication No. WO 2005/058886, and EP 1,683,803. Following methods of these publications and references cited therein, respective prodrugs of the compounds of provided herein can be likewise prepared. Thus, prodrugs of compounds of the formula I are prepared by modifying functional groups present in a compound provided herein in such a way that the modifications may be cleaved in vivo to release the parent compound. Said prodrugs can be used, for example, to improve aq. solubility, oral, transdermal, or ocular bioavailability, to achieve a controlled (e.g., extended) release of the drug moiety, to improve tolerability, etc. Prodrugs include compounds disclosed herein wherein a hydroxy, sulfhydryl, amido or amino group in the compound is bonded to any group that may be cleaved in vivo to regenerate the free hydroxyl, amido, amino, or sulfhydryl group, respectively. Examples of prodrugs include, but are not limited to esters (e.g., acetate, formate, benzoate, phosphate or phosphonate derivatives), carbamates (e.g., N,N-dimethylaminocarbonyl), N-phosphoramides, of hydroxyl or amine-derived functional groups in compounds provided herein. Prodrug derivative can be used either as a neutral prodrug form (e.g., acid or amine), or a respective salt form thereof [e.g., sodium salt of a phosphate prodrug, or an amine salt (e.g., hydrochloride, citrate, etc.) for an amine group-bearing prodrug], or a zwitterionic form if both positively and negatively charged/ionizable functions are present. Prodrug groups may be incorporated at various sites of the formula I.

The compounds disclosed herein are generally named according to the IUPAC or CAS nomenclature system. Abbreviations which are well known to one of ordinary skill in the art may be used (e.g., “Ph” for phenyl, “Me” for methyl, “Et” for ethyl, “h” for hour or hours and “r.t.” for room temperature).

### General Synthetic Methods

Novel methods for the preparation or manufacture of antibacterial 1-(ortho-fluorophenyl)dihydropyridone oxazolidinones are provided herein. Synthesis of aforementioned oxazolidinones may in part follow some known in the heterocyclic chemistry methods generally described for certain des-ortho-fluorophenyl heterocyclic derivatives (i.e., those lacking the ortho-F group). To achieve the novel ortho-fluorophenyl substitution pattern in a compound provided herein, an aromatic reagent generally described for preparation of des-ortho-fluorophenyl heterocyclic compounds may be intentionally replaced for a specific reagent containing at least one appropriately positioned ortho-F substituent. Because of the different reactivity pattern for many ortho-F compounds, novel methods for synthesis and manufacturing must be deployed.

Specific inventive steps of the present application provide for novel efficient methods and processes for the synthesis and manufacturing of the key intermediates and the target 1-(ortho-fluorophenyl)dihydropyridone oxazolidinones.

In one aspect, the synthesis of the compounds disclosed herein are illustrated in general Scheme 1. Oxazolidinone-forming reagents similar to those employed at the step (c) of the Scheme 1 have been more generally described in Org. Proc. Res. & Development, 2003, p. 533. Extension of these methods may include new manipulations to remove optional other protective groups, if different from tert-butoxycarbonyl (Boc) group illustrated in Scheme 1.

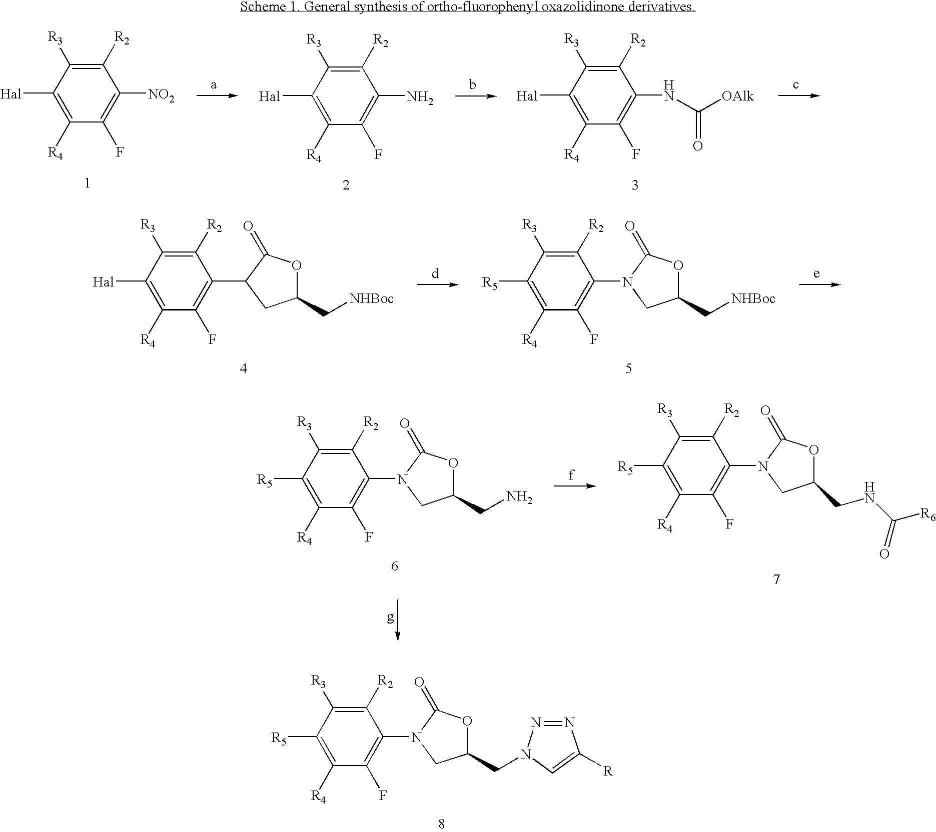

a) Reducing reagent(s): e.g., H₂, Pd/C, Fe/NH₄Cl, or SnCl₂ etc.; b) Carbamate-forming reagent: e.g., AlkOC(═O)Cl, AlkOCOC₆F₅, or alike; base: NaOH, NaH, Py, triethylamine (TEA) or alike; c) oxazolidinone-forming reagent(s): e.g., (s)-tent-butyl 3-chloro-2-hydroxypropylcarbamate, or (S)-tert-butyl oxiran-2-ylmethylcarbamate; base: LiOBu-t, KOBu-t, NaH, or alike; d) arylating or heteroarylating reagent(s): e.g., Ar—B(OH)₂, Ar—B(OAlk′)₂, Het¹-B(OH)₂, Het¹-B(OAlk′)₂, Het²-B(OH)₂, or Het²-B(OAlk′)₂ selected from boronic acid, boronic acid ester (e.g., (picolinato)boron ester or alike, Pd catalyst (e.g., PdCl₂(dppf)DCM, Pd(PPh₃)₄ or alike); e) acid (e.g., TFA or HCl solution in organic solvent, e.g., THF or dioxane), base (e.g., NaHCO₃, TEA, or alike); 0 acylating agent: e.g., R⁶C(═O)Cl, R⁶C(═)OC₆F₅, or R⁶COOH/HATU; base: K₂CO₃, TEA or alike; g) triazole-forming reagent: e.g., TsNHN═C(CHCl₂)Alk; base: e.g., K₂CO₃, TEA, or alike.

Analogously to the step (d) of Scheme 1, various heterocyclic derivatives have been prepared by metal-mediated transformations of 4-halo-phenyl heterocyclic derivatives as more generally described, for example, in International Patent Publication Nos. WO 1999/064417, 2005/012271, and WO 2005/058886, each of which is incorporated herein by reference in its entirety. Likewise, boron-coupling chemistry of step (d) may be optionally supplanted by other metal-mediated couplings, such as tin-coupling chemistry similar to that described more generally in WO 2005/012271, incorporated herein by reference in its entirety.

Additional general routes to the compounds disclosed herein are illustrated in Scheme 2. Mitsunobu alkylation chemistry of step (c) is precedented in analogous heterocyclic chemistry more generally described, for example, in International Patent Publication No. WO 1999/064416, incorporated herein by reference in its entirety. Triazole-forming chemistry analogous to that of step (e) of this scheme has been generally described, for example, in Heterocycles, 1998, p. 895, and in Org. Lett., 2008, p. 497.

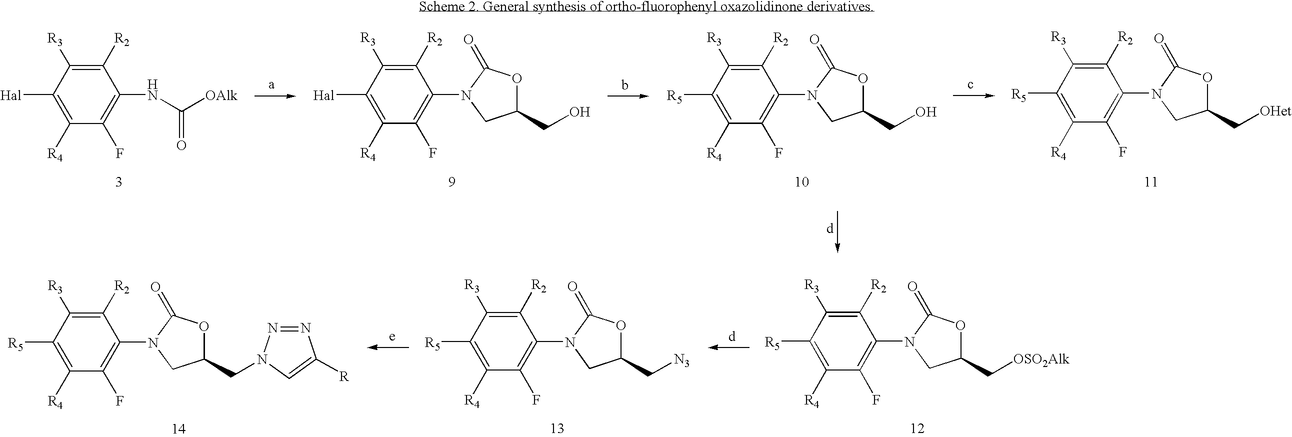

a) Oxazolidinone-forming reagent(s): (R)-glycidyl butyrate, (R)-glycidol, or alike; base: BuLi, lithium hexamethyldisilylamide (LHMDS), LiOBu-t, KOBu-t, NaH, or alike; b) arylating or heteroarylating reagent(s): e.g., Ar—B(OH)₂, Ar—B(OAlk′)₂, Het¹-B(OH)₂, Het¹-B(OAlk′)₂, Het²-B(OH)₂, or Het²-B(OAlk′)₂ selected from boronic acid, boronic acid ester (e.g., (picolinato)boron ester) or alike, Pd catalyst (e.g., PdCl₂(dppf)DCM, Pd(PPh₃)₄ or alike); c) Het¹OH or Het²OH, Mitsunobu reagents: e.g., triphenylphosphine, DIAD, base; d) RSO₂Cl, base; e) azide-forming reagent: NaN₃, LiN₃, or alike; f) triazole-forming reagent: e.g., R—C≡C—H, norbornadiene, or alike.

Another general route to compounds disclosed herein featuring an (isoxazole-3-yl)ylamino group is illustrated in Scheme 3 below.

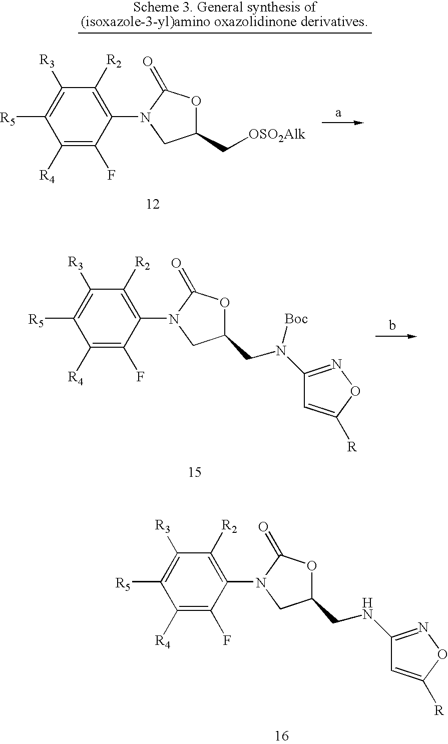

a) 3-(N-Boc-amino)-5-R-isoxazole; base: e.g., NaH, LiOBu-t, KOBu-t, tetramethylguanidine, or alike; c) acid: TFA or HCl solution in organic solvent, e.g., THF or dioxane); then base: NaHCO₃, TEA, or alike.

Optionally, the substituent R⁵ can be installed into the requisite phenyl reagent prior to the oxazolidinone formation. Some methods for synthesis of a dihydropyridone group derivatives have been generally described, for example, in publications Tetrahedron Lett., 1973, p. 5095; Tetrahedron Lett., 1991, p. 3643; Tetrahedron Lett., 1995, p. 3985; Tetrahedron Lett., 1995, p. 9449; Heterocycles, 1997, p. 57, Tetrahedron Lett., 1997, p. 7565.

Once the desired group R⁵ is installed, the synthesis can be completed by either by general methods of any one of Schemes 1-3 or variations thereof, except that no coupling step to replace the Hal group for R⁵ group is required (i.e., R⁵ instead of Hal in the intermediate 1 of Scheme 1). For example, if the R⁵ group is a dihydropyridone group, then compounds of formula I are obtained.

Additional methods for the preparation or manufacture of the compounds of formula I provided herein are illustrated in Scheme 4 below.

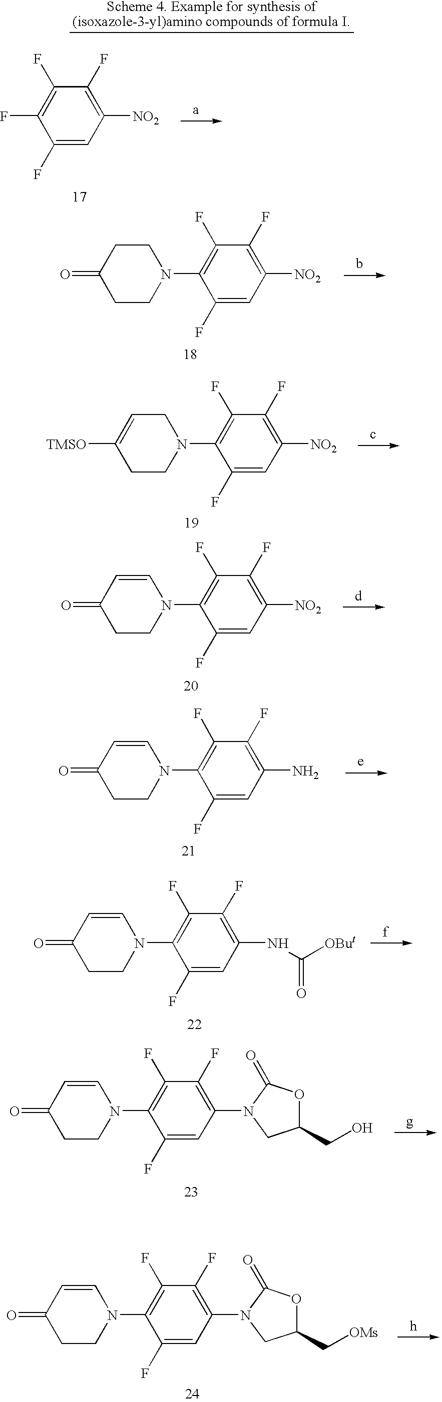

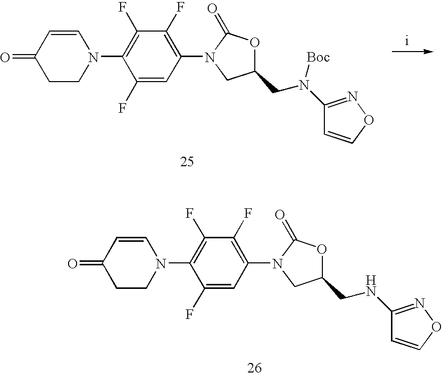

a) Piperidin-4-one hydrochloride, DIEA, NMP, −5° C. to r.t.; b) TMSOTf, TEA, THF, 0° C. to r.t.; c) O-allyl-O′-methyl carbonate, Pd(OAc)₂, DMSO, 2,3,4,5-tetrafluoronitrobenzene, 60° C.; d) Fe, NH₄Cl, EtOH, 95° C.; e) isobutyl chloroformate, Py, DCM, 0° C. to r.t.; f) two steps: 1) (R)-glycidyl butyrate or chlorohydrine, Bu^{t}OLi, THF, MeCN, 0-30° C.; 2) 10% aq. K₂CO₃; g) MsCl, TEA, THF, 0° C.; h) 3-[N-(tert-butoxycarbonyl)amino]isoxazole, Bu^{t}OK, DMF, 20-40° C.; i) aq. HCl, EtOH, EtOAc, 0° C. to r.t.

Select innovative steps pertaining to the particular utility of Scheme 4 for an efficient synthesis and production of the compounds of formula I (illustrated by structure 26 in the Scheme 4) are summarized in paragraphs (i-iv) below:

i) The novel efficient method for an installation of the dihydropyridone ring into an ortho-F compound of formula I provided herein involve the use of an alkoxide (e.g, methoxide) capture reagent (e.g., 2,3,4,5-tetrafluoronitrobenzene). The dihydropyridone-forming step for a transformation of the compounds 19 to compounds 20 performed in absence of the methoxide-capture reagent(s) is accompanied by formation of the hard-to-remove ortho-methoxy impurity (e.g., 1-(2,6-difluoro-3-methoxy-4-nitrophenyl)-2,3-dihydropyridone) resulted from undesired substitution of ortho-F atom with MeOH, AlkOH, or anion thereof. This is a serious problem specific for the synthesis of ortho-F dihydropyridone compounds, arising from the unique reactivity of ortho-F substrates 19 and may not be encountered in synthesis of des-ortho-F compounds lacking the key ortho-F substitution. The methods disclosed herein involve the use of a methoxide-capture nitrobenzene additive to eliminate or minimize above methoxy-aryl by-product to allow for a high-yielding preparation and manufacture of precursors 19 and compounds of formula I, with a purity suitable for pharmaceutical applications (generally, better than 90-95%). Additional MeO-capture additives may include acylating, alkylating, or arylating agents (e.g., carboxylic acid anhydride or an active ester capable of methoxide acylation). Optionally, one or more alkoxide-capture reagent(s), or a combination thereof can be used.

ii) New practical method for the key oxazolidinone-forming step (from 22 to 23) provided herein involves the use of an alkali metal alkoxide (e.g., LiOBu-t) instead of the conventionally used BuLi (as more generally described, e.g., in J. Med. Chem., 1988, vol. 41, pp. 3727-3735). The procedure provided herein thus eliminates the use of a highly flammable and unstable organometallic chemical. Moreover, the new processes provided herein also eliminates the need for costly cryogenic (−78° C.) conditions impractical for the industrial manufacture of the reagents 23 and of the compounds of formula I.

iii) Novel process for the preparation of 5-[(isoxazole-3-yl)amino]methyl derivatives 25 that employs an alkali metal alkoxide (e.g., KOBu-t) in place of previously used NaH (as more generally described, e.g., in International Patent Publication No. WO 00/21960, incorporated herein by reference in its entirety). This eliminates the use of an extremely flammable base and allows for an efficient preparation and manufacture of the precursors 25 and the compounds of formula I.

iv) New practical method for the synthesis of the compounds of formula I (R₁=(isoxazole-3-yl)amino; structure 26 in Scheme 4) employing aq. HCl-organic solvent(s) system for deprotection of acid-cleavable protective groups (PG; e.g., PG=tert-butoxycarbonyl or Boc group). The method provided herein eliminates the use of highly toxic and expensive reagents conventionally employed for des-ortho-F 1-phenyldihydropyridone compounds (the method as described, for example, in International Patent Publication No. WO 2004/033449, advocating the use of trifluoroacetic acid and 1,2-dichloroethane Boc-deprotection system). The efficiency of the new deprotection method invented herein is particularly surprising in view of the fact that enamino ketones (such as dihydropyridones) are generally degradable by a strong aqueous acids, such as aq. HCl (as more generally described, e.g., by Katritzky et al. in J. Chem. Research, Miniprint, 1980, pp. 3337-3360).

Additional new procedures and detailed synthetic schemes for the synthesis and manufacture of specific compounds disclosed herein are further illustrated by methods described for Examples below.

### EXAMPLES

Methods and processes provided herein are described in the following examples, which are meant to illustrate and not limit the scope of this invention. Common abbreviations well known to those with ordinary skills in the synthetic art used throughout. ¹H NMR spectra (δ, ppm) are recorded in CDCl₃ unless specified otherwise. Mass-spectroscopy data for a positive ionization method are provided. Chromatography means silica gel chromatography unless specified otherwise. TLC means thin-layer chromatography. Unless specified otherwise, all reagents were either from commercial sources, or made by conventional methods described in available literature.

### Example 1

### Compound of Structure

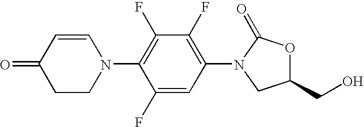

Scheme for the Compound of Example 1:

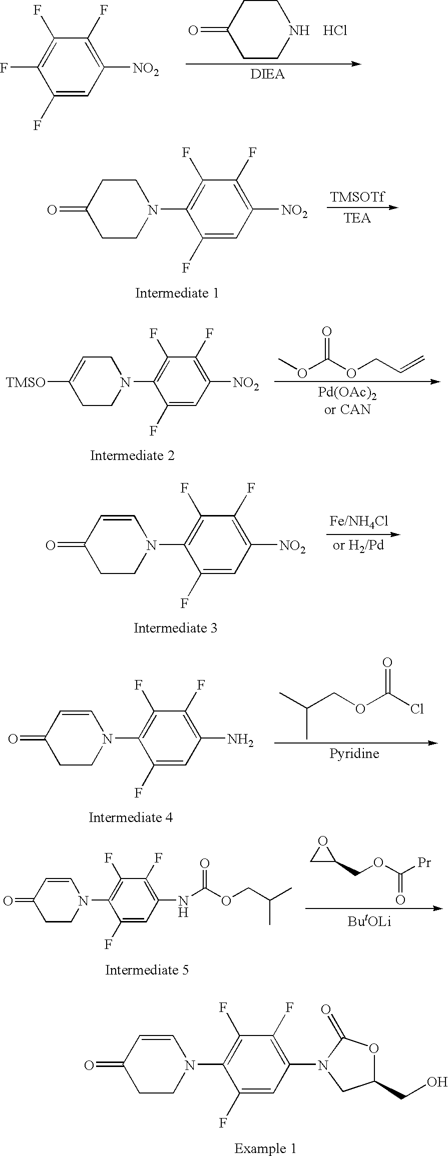

Intermediate 1. In a 1 L flask was added 4-piperidone hydrochloride (82.0 g, 534 mmol), 2,3,4,5-tetrafluoronitrobenzene (94.7 g, 486 mmol) and NMP (110 mL). The solution was cooled to 4° C., and DIEA (156.8 g, 200 mL, 71.2 mmol) was added slowly with stirring. The temperature was maintained below 10° C. during the addition. The reaction mixture was warmed up to r.t. and stirred overnight, monitoring the consumption of 2,3,4,5-tetrafluoronitrobenzene by TLC (25% EtOAc/petroleum ether). The reaction mixture was poured slowly into 1.5 L of ice water with stirring. Yellow solid precipitated was filtered, washed with water (ca. 5×) and dried under vacuum (oil pump) at 68° C. for 5 h. The yellow solid thus obtained (140 g, 90%) was used for the next step without further purification. ¹H NMR (400 MHz, CDCl₃): 7.74 (m, 1H); 3.73 (t, J=6.0 Hz, 4H); 2.66 (t, J=6.0 Hz, 4H). ¹H NMR (400 MHz, CDCl₃): 7.74 (m, 1H); 3.73 (t, J=6.0 Hz, 4H); 2.66 (t, J=6.0 Hz, 4H). MS (m/z): 275 [M+H].

Intermediate 2. Trimethylsilyl trifluoromethanesulfonate (69.3 g, 310 mmol) was added dropwise with stirring at 0° C. to Intermediate 1 (71.2 g, 260 mmol) and triethylamine (52.6 g, 260 mmol) in 300 mL of THF under nitrogen. The reaction mixture was allowed to warm up to r.t. and stirred ca. 1 h (until the starting material disappeared). Most of the solvent was removed and the reaction mixture was quenched with water (600 mL) and extracted with petroleum ether (3×500 mL). The organic layers were combined and washed with brine (500 mL). The organic layer was dried over Na₂SO₄, filtered and concentrated to afford the crude product as yellow oil that solidified upon cooling to −20° C. The solid was dried under high vacuum overnight (88.5 g, 98%). ¹H NMR (400 MHz, DMSO-d₆): 7.96 (m, 1H); 4.92 (brs, 1H); 3.92 (m, 2H); 3.50 (t, J=5.4 Hz, 2H); 2.21 (m, 2H); 0.20 (s, 9H).

Intermediate 3.

Method A. To a solution of Intermediate 2 (100 g, 288 mmol) and allyl methyl carbonate (39 mL, 1.4 eq.) in 600 mL of dry DMSO was added 2,3,4,5-tetrafluoronitrobenzene (17.4 mL, 0.5 eq.) and Pd(OAc)₂ (4.93 g, 20.2 mmol, 0.07 eq.). The reaction mixture was heated for 1.5 h at 60° C. Gas evolution (CO₂) was observed. The reaction mixture was stirred until the starting material disappeared, then it was poured into 1 L of ice water. Petroleum ether (1 L) was added and the mixture was stirred for 3-10 h at r.t. The organic layer was separated and ethyl acetate was added to dissolve the solid. The mixture was then passed through a short silica gel-celite column and washed with ethyl acetate. The ethyl acetate layer was separated, and water layer was extracted again with ethyl acetate (2×500 mL). The combined organic layers were washed with brine (3×500 mL), dried over Na₂SO₄, filtered and concentrated. The yellow oil was dried under vacuum. The desired product was obtained as a yellow solid (75 g, 95%).

Method B. Ceric ammonium nitrate (CAN, 19.0 g, 34.65 mmol) was added portionwise with stirring to a solution of the Intermediate 2 (12.4 g, 28.80 mmol) in DMF (100 mL) at 0° C. The reaction mixture was allowed to warm up to r.t. and stirred for another 4 h. Most of solvent was removed under vacuum. Water (ca. 75 mL) was added and the mixture was extracted with EtOAc (2×100 mL). The combined organic layers were washed with brine and dried (Na₂SO₄). Solvent was removed and the residue purified by column chromatography (gradient 20% to 30% EtOAc in petroleum ether). The product was obtained as a yellow solid. ¹H NMR (400 MHz): 7.84 (m, 1H); 7.14 (m, 1H); 5.43 (d, J=8.2 Hz, 1H); 4.06 (t, J=7.2 Hz, 2H); 2.74 (t, J=7.2 Hz, 2H). MS (m/z): 273 [M+H].

Intermediate 4.

Method A. Intermediate 3 (49.1 g, 0.18 mol) was dissolved in a mixed solvent of ethanol (500 mL) and water (150 mL). Solid NH₄Cl (57.6 g, 1.08 mol) was added, followed by iron powder (50 g, 0.9 mol). The mixture was heated at 80° C. for 1 h with vigorous stirring. After cooling to r.t., the mixture was passed through a short celite pad to remove the iron residue. The flask and celite pad were washed with ethanol. The filtrate was condensed under vacuum and most of the ethanol was removed. Water (200 mL) was added and the mixture was extracted with EtOAc (2×400 mL). The combined organic layers were washed with brine and dried over Na₂SO₄. Solvent was removed and the product was obtained as a brown solid and was used directly for the next step.

Method B. To a solution of Intermediate 3 (50 mg, 0.18 mmol) in MeOH (2 mL) was added Pd/CaCO₃ (5 mg, 10 wt %), followed by acetic acid (0.02 mL). The mixture was hydrogenated by putting a balloon filled with hydrogen on top of the flask. The reaction mixture was heated at 40° C. overnight. TLC showed starting materials disappeared. After cooling down to r.t., the mixture was filtered and the catalyst cake was washed with MeOH (2 mL). The filtrate was concentrated and the residue was used directly for the next step without further purification. Quantitative yield based on NMR for the crude material. ¹H NMR (400 MHz, CDCl₃): 7.03 (m, 1H); 6.36 (m, 1H); 5.19 (d, J=8.0 Hz, 1H); 4.12 (d, J=7.2 Hz, 2H); 3.80 (t, J=7.2 Hz, 2H); 2.66 (t, J=7.2 Hz, 2H). MS (m/z): 243 [M+H].

Intermediate 5. A 250 mL flask was charged with Intermediate 4 (10.0 g, 41 mmol), pyridine (4.0 mL, 50 mmol) and DCM (160 mL). Isobutyl chlorofomate (5.8 mL, 45 mmol) was added slowly with stirring at 0° C., and the mixture was warmed up to r.t. and stirred for 90 min. The reaction was quenched with water and the organic layer was separated. The water layer was washed with DCM (100 mL). The combined DCM was washed with 5% aq. NaHCO₃ (150 mL), brine and dried over Na₂SO₄. After filtration the filtrate was condensed and the residue was further purified by passing through a short silica column (ca. 30 g silica gel) and washed with a solution of 1% MeOH in DCM. The filtrate was condensed and the desired product was obtained as a light yellow solid (13.1 g, 92%). ¹H NMR (400 MHz, DMSO-d₆): 9.93 (s, 1H); 7.59 (m, 1H); 7.42 (d, J=7.6 Hz, 1H); 5.0 (d, J=8.0 Hz, 1H); 3.89 (d, J=6.8 Hz, 2H); 3.81 (t, J=7.2 Hz, 2H); 2.47 (t, J=8.0 Hz, 2H); 1.91 (m, 1H). MS (m/z): 343 [M+H].

### Compound of Example 1

Intermediate 5 (17.0 g, 49 mmol) was dissolved in anhydrous THF (10 mL) and CH₃CN (34 mL) at 5° C. under nitrogen. Bu^{t}OLi (powder, 4.3 g, 54 mmol) was added slowly and the resulting solution was stirred for another 1 h at 5° C. (R)-Glycidyl butyrate (14.1 mL, 99 mmol) was added dropwise. The mixture was stirred for 3 h and allowed to warm up to 17-20° C. The reaction mixture was cooled to 5° C. and quenched carefully with aq. acetic acid solution (1.0 M, 85 mL). The solid was dissolved after stirring for 3 h at 5° C. The mixture was condensed under vacuum, and the residue was re-dissolved in DCM (100 mL) and washed with water (50 mL). The DCM phase was condensed under vacuum. The residue was dissolved in CH₃OH (34 mL) and cooled to 10° C. Then 10% aq. K₂CO₃ solution (30 mL) was added slowly. The mixture was warmed up to 20° C. and stirred for 2 h. After cooling to 5° C., aq. acetic acid solution (1.0 M, 50 mL) was added dropwise to adjust the pH to ca. 6-7. The mixture was extracted with DCM (3×60 mL). The combined DCM layers were washed with brine (80 mL) and 1% aq. HCl (30 mL), dried over MgSO₄, filtered and condensed on vacuum. The solid obtained was washed with a mixture of EtOAc (15 mL) and heptane (15 mL) for 2 h at 75° C., filtered and dried. The product was obtained as an off-white solid (11.1 g, 65%). ¹H NMR (400 MHz, DMSO-d₆): 7.55 (m, 1H); 7.46 (d, J=7.6 Hz, 1H); 5.24 (t, J=4.8 Hz, 1H); 5.04 (d, J=8.0 Hz, 1H); 4.75 (m, 1H); 4.09 (t, J=4.8 Hz, 1H); 3.86 (t, J=7.2 Hz, 3H); 3.67 (m, 1H); 3.55 (m, 1H); 3.30 (s, 2H). MS (m/z): 343 [M+H].

### Example 2

### Compound of Structure

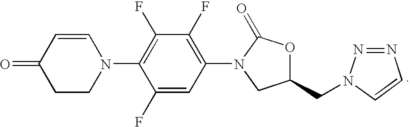

Scheme for Compound of Example 2:

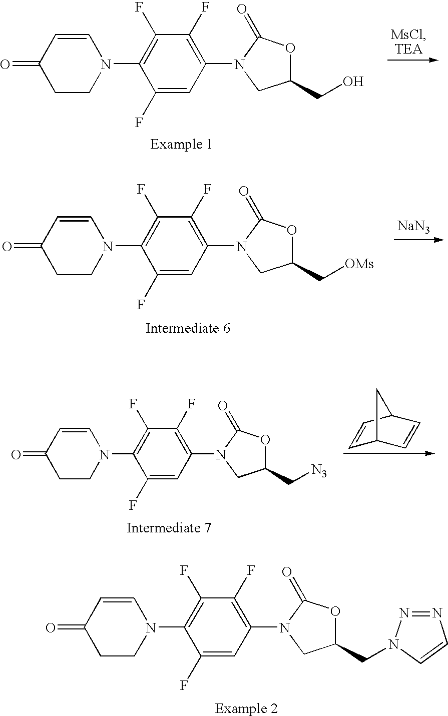

Intermediate 6. To a suspended solution of the compound of Example 1 (12.6 g, 36.8 mmol) and TEA (15.4 mL, 110.4 mmol) in DCM (150 mL) was added MsCl (5.7 mL, 73.6 mmol) dropwise at 0° C. After stirring for 1 hour at 0° C., the reaction mixture was taken into brine and the DCM layer was collected. The DCM layer was washed with saturated NH₄Cl (100 mL) and the combined organic layers were washed with brine (100 mL) again, and dried over MgSO₄. Most of the solvent was removed under vacuum. The moist solid was filtered, crushed and suspended in EtOAc (30 mL). After stirring for 1 h at 15° C., the suspension was filtered and dried under vacuum at ≦60° C. The product was obtained as an off-white solid (11.6 g, 75%). ¹H NMR (400 MHz, CDCl₃): [symbol] 7.40 (m, 1H); 7.10 (d, J=7.6 Hz, 1H); 5.33 (d, J=8.0 Hz, 1H); 5.00 (m, 1H); 4.53 (dd, J=12.0, 3.2 Hz, 1H); 4.45 (dd, J=12.0, 3.6 Hz, 1H); 4.29 (t, J=9.2 Hz, 1H); 4.04 (dd, J=8.8, 1.6 Hz, 1H); 3.93 (t, J=7.2 Hz, 2H); 2.70 (t, J=7.4 Hz, 2H); 3.15 (s, 3H).

Intermediate 7. A mixture of the Intermediate 6 (567 mg, 1.35 mmol) and NaN₃ (438 mg, 6.75 mmol) in DMF (5 mL) was stirred at 55° C. o.n. After cooling to r.t., water (15 mL) was added, and the reaction mixture was extracted with DCM (3×30 mL). Combined organic layers were washed with brine (30 ml) and dried (Na₂SO₄). Solvent was removed under vacuum to afford the product as a light yellow solid. This was used directly for the next step without further purification.

### Compound of Example 2

A mixture of the Intermediate 7 (785 mg, 2.14 mmol) and bicyclo[2.2.1]hepta-2,5-diene (2.2 mL, 21.4 mmol) in 1,4-dioxane (22 mL) under N₂ was heated at 100° C. for 3 h. Most of volatiles were removed under vacuum, and the residue was purified by column chromatography (1% MeOH/DCM). Thus isolated product was recrystallized from MeOH and collected as a white solid. ¹H NMR (400 MHz): 7.83 (s, 2H), 7.05 (m, 2H), 5.30 (d, J=8 Hz, 1H), 5.16 (m, 1H), 4.83 (d, J=3.6 Hz, 2H), 4.33 (m, 1H), 4.06 (m, 1H), 3.91 (t, J=14.8 Hz, 2H), 2.69 (t, J=14.8 Hz, 2H). MS (m/z): 394 [M+H].

### Example 3

### Compound of Structure

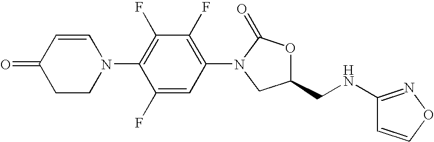

Scheme for Compound of Example 3:

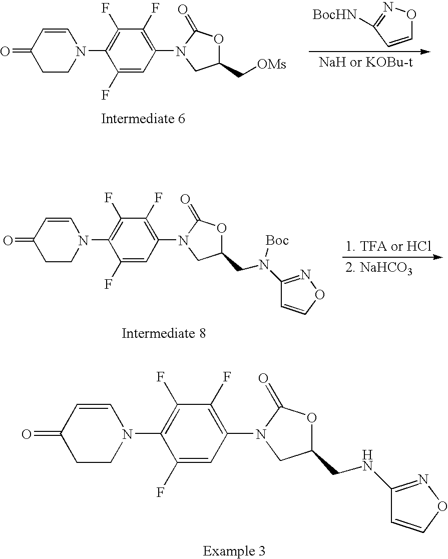

Intermediate 8.

Method A. A solution of tert-butyl isoxazol-3-ylcarbamate (187 mg, 1.00 mmol) in DMF (1 mL) was added dropwise with stirring to a suspension of NaH (60% in mineral oil, 48 mg, 1.20 mmol) in DMF (2 mL). The mixture was stirred under N₂ for 15 min. at 35° C. The Intermediate 6 (357 mg, 0.85 mmol) in DMF (1 mL) was added, and the mixture was stirred at 50° C. for 1.5 h. The reaction mixture was taken into EtOAc (30 mL), washed with 10% aq. NH₄Cl (2×15 mL), brine, and dried (Na₂SO₄). Solvent was removed under vacuum and the crude material was purified by column chromatography (2% MeOH/DCM) to afford the product as a light yellow solid.

Method B. A solution of tent-butyl isoxazol-3-ylcarbamate (13.2 g, 67.2 mmol) in DMF (30 mL) was added to a mixture of Bu^{t}OK (16.8 g, 64 mmol) in DMF (60 mL) at 5° C. The mixture was then warmed up to 20° C. and stirred for 1 h. The resulting solution was added to a mixture of Intermediate 6 (13.4 g, 32 mmol) in DMF (60 mL) and heated at 40° C. for 3 h. Additional reagent pre-made as above from tert-butyl isoxazol-3-ylcarbamate and Bu^{t}OK (6.4 mmol, 0.2 eq) was added, and the mixture was stirred for 2 h. The latter process was repeated again until Intermediate 6 was consumed. The reaction mixture was cooled to r.t. and poured into water (300 mL) and extracted with EtOAc (3×200 mL). The combined organic layers were washed with 10% aq. NaCl (3×300 mL), brine (300 mL), and dried over MgSO₄. The solution was filtered and the filtrate was condensed under vacuum. The residue was recrystallized from EtOH (20 mL) and water (18.5 mL). The collected solid was suspended in methyl tert-butyl ether (MTBE, 50 mL) and stirred for 4 h. The product was collected as a pale yellow solid (12 g, 75%). ¹H NMR (400 MHz, CDCl₃): 8.28 (s, 1H); 7.44 (m, 1H); 7.09 (d, J=7.6 Hz, 1H); 7.00 (s, 1H); 5.32 (d, J=7.6 Hz, 1H); 5.15 (m, 1H); 4.44 (m, 1H); 4.20 (m, 2H); 3.94 (m, 3H); 2.70 (t, J=7.4 Hz, 2H); 1.45 (s, 9H). MS (m/z): 509 [M+H].

### Compound of Example 3

Method A. TFA (2.0 mL) was added dropwise to the solution of the Intermediate 8 (310 mg, 0.61 mmol) in 1,2-dichloroethane (DCE; 2 mL) at 0° C., and the solution was stirred at 0° C. for 30 min. Volatiles were removed under vacuum, and the residue taken into EtOAc (30 mL). The solution was washed with saturated NaHCO₃ solution (2×15 mL), brine, and dried (Na₂SO₄). Solvent was removed under vacuum and the crude product was purified by column chromatography (3% MeOH/DCM). Light-yellow solid.

Method B. Intermediate 8 (16.0 g, 31.6 mmol) was suspended in a mixture of EtOH (40 mL) and EtOAc (40 mL) and cooled to 0° C. Conc. HCl (60 mL) was added dropwise, and the suspension turned clear. The mixture was warmed up to 20° C. and stirred for 2 h. The mixture was cooled down to 5° C. and neutralized with 10% aq. NaOH to pH ca. 4-5. Then saturated aq. Na₂CO₃ (ca. 25 mL) was added to bring the pH to ca. 8.0. The mixture was filtered, and the filtrate was condensed under vacuum. The precipitated solid was collected, and re-dissolved in 95% EtOH (100 mL) with heating to ca. 80° C. After cooling to 55° C., activated carbon (2.5 g) was added, and the mixture was stirred for 3 h at 55° C. The warm solution was filtered, and the activated carbon was washed several times with warm EtOH. The filtrated was condensed under reduced pressure. The solid thus obtained was recrystallized with EtOH (30 mL) and water (20 mL). The final product was collected as a pale yellow solid (8.2 g, 65%). ¹H NMR (400 MHz, DMSO-d₆): 8.41 (d, J=1.6 Hz, 1H); 7.57 (m, 1H); 7.50 (d, J=8.0 Hz, 1H); 6.58 (t, J=5.8 Hz, 1H); 6.02 (d, J=1.6 Hz, 1H); 5.08 (d, J=8.0 Hz, 1H); 4.90 (m, 1H); 4.17 (t, J=8.6 Hz, 1H); 3.86 (m, 3H); 3.48 (t, J=5.6 Hz, 2H); 2.49 (overlapped with DMSO-d₆, 2H). MS (m/z): 409 [M+H].

### Example 4

### Compound of Structure

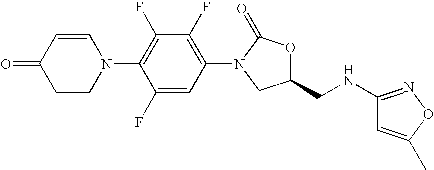

Scheme for Compound of Example 4:

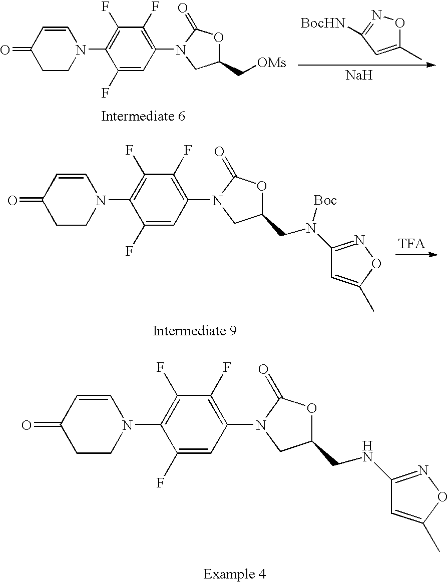

Intermediate 9. NaH (60% in mineral oil, 7 mg, 0.18 mmol) was added with stirring to tert-butyl 5-methylisoxazol-3-ylcarbamate (34 mg, 0.17 mmol) in DMF (1 mL) at 0° C. The mixture was stirred at this temperature for 15 min, and then at 35° C. for 30 min. The Intermediate 6 (60 mg, 0.14 mmol) in DMF (1.00 mL) was added, and the mixture was stirred at 50° C. for 1.5 h. The reaction mixture was taken into EtOAc (30 mL), washed with 10% aq. NH₄Cl (2×15 mL), brine, and dried (Na₂SO₄). Solvent was removed under vacuum and the crude product was purified by column chromatography (2% MeOH/DCM) to afford the product that was used for the next step without purification.

### Compound of Example 4

The synthetic step was performed just as described for the Compound of Example 3, except using the Intermediate 8 from above step instead of the Intermediate 9. The crude product was purified by preparative TLC (5% methanol/DCM). Light-yellow solid. ¹H NMR (400 MHz, DMSO-d₆): 7.57 (m, 1H), 7.49 (d, J=8.0 Hz, 1H), 6.47 (t, J=6.0 Hz, 1H), 5.70 (s, 1H), 5.07 (d, J=8.0 Hz, 1H), 4.92 (m, 1H), 4.16 (t, J=8.8 Hz, 1H), 3.87 (m, 3H), 3.43 (t, J=5.6 Hz, 2H). MS (m/z): 423 [M+H].

### Example 5

### Compound of Structure

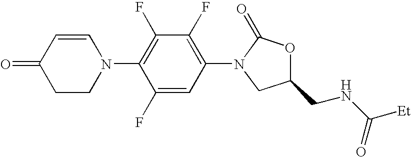

Scheme for Compound of Example 5:

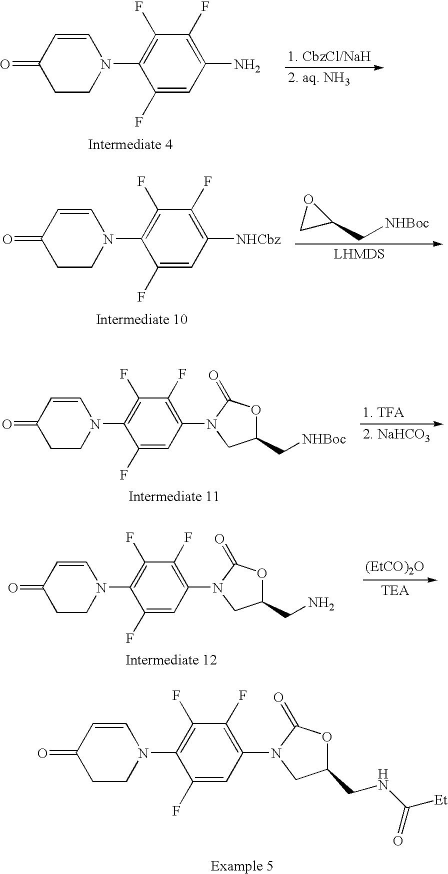

Intermediate 10.60% NaH in mineral oil (1.4 g, 36.0 mmol) was added portionwise with stirring to the Intermediate 20 (2.9 g, 11.94 mmol) in THF (20 mL) at 0° C. under Ar, and the mixture was stirred at this temperature for 30 min. Benzyl chloroformate (4.1 g, 24.03 mmol) was added dropwise with stirring. The reaction mixture was allowed to warm up to r.t. and stirred o.n. The reaction was carefully quenched with water (10 mL), and THF was removed under vacuum. The residue was taken in DCM (80 mL). Organic layer was washed with brine (50 mL) and dried (Na₂SO₄). Solvent was removed under vacuum, and the residue dissolved with MeOH (40 mL). Aq. NH₃ (25 mL) was added with stirring, and the mixture was stirred at r.t. for 2 h. Solvent was removed under vacuum, and EtOAc (100 mL) was added. The organic layer was washed with brine and dried (Na₂SO₄). Solvent was removed under vacuum, and the residue purified by column chromatography (gradient 25% to 100% DCM/petroleum ether). White solid. ¹H NMR (400 MHz): 7.95 (m, 1H); 7.41 (m, 6H); 7.07 (m, 2H); 5.28 (s, 2H); 3.88 (t, J=7.6 Hz, 2H); 2.69 (t, J=7.6 Hz, 2H). MS (m/z): 377 [M+H].

Intermediate 11. 1.06M Lithium hexamethyldisilylamide in THF (LHMDS; 0.45 mL, 0.48 mmol) was added dropwise with stirring to a solution of the Intermediate 10 (151 mg, 0.40 mmol) in THF (2 mL) under N₂ at −78° C. After ca. 30 min, a solution of (S)-tert-butyl oxiran-2-ylmethylcarbamate (139 mg, 0.80 mmol) in THF (1.5 mL) was added dropwise with stirring. The mixture was allowed to warm up to r.t. and stirred o.n. Saturated aq. NH₄Cl solution (10 mL) was added, and the solution extracted with EtOAc (3×10 mL). Combined organic layers were washed with brine and dried (Na₂SO₄). The product was isolated by preparative TLC (95% DCM/MeOH) as yellow oil that was used directly for the next step.

Intermediate 12. TFA (0.2 mL) was added to the Intermediate 11 (102 mg, 0.23 mmol) in DCE (2 mL) at 0° C., and the solution was kept at this temperature for ca. 15 min. The reaction was quenched with 5% aq. NaHCO₃ and extracted with DCM (2×10 mL). The combined organic layers were dried (Na₂SO₄), and the solvent was removed in vacuo to afford the product as a pale yellow solid.

### Compound of Example 5

TEA (139 μL, 1.0 mmol) was added to a solution of the Intermediate 11 in DCM (2 mL) at 0° C., followed by propionic anhydride (52 μL, 0.40 mmol). The reaction mixture was stirred at 0° C. for 30 min. Water (2 mL) was added, and the mixture extracted with DCM (3×5 mL). Combined organic layers were washed with brine and dried (Na₂SO₄). The crude material was purified by preparative TLC (5% MeOH/DCM) to afford the product as a white solid. ¹H NMR (400 MHz): 7.31 (m, 1H); 7.07 (d, J=7.6 Hz, 1H); 6.36 (t, J=12.4 Hz, 1H); 5.29 (d, J=8.0 Hz, 1H); 4.86 (m, 1H); 4.15 (t, J=17.6 Hz, 1H); 3.91 (t, J=14.8 Hz, 3H); 3.70 (m, 2H); 2.69 (t, J=15.2 Hz, 2H); 2.30 (m, 2H); 3.21 (t, J=14.8 Hz, 3H). MS (m/z): 398 [M+H].

### Example 6

### Compound of Structure

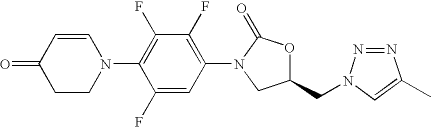

Scheme for Compound of Example 6:

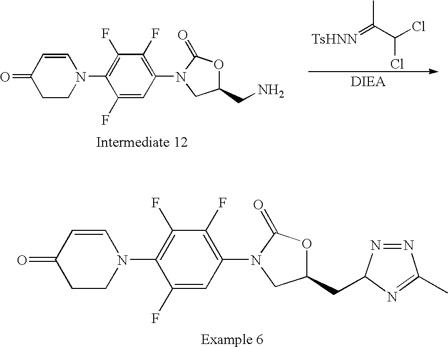

### Compound of Example 6

N′-(1,1-Dichloropropan-2-ylidene)-4-methylbenzenesulfonohydrazide (106 mg, 0.36 mmol) was added with stirring to a solution of the Intermediate 12 (82 mg, 0.24 mmol) and DIEA (200 μL, 1.2 mmol) in MeOH (1 mL) under Ar at 0° C. The reaction mixture was allowed to warm up to r.t. and stirred for 3 h. The solvent was removed under vacuum and the residue taken into DCM. Resulting mixture was washed with water and dried (Na₂SO₄). The filtrate was concentrated under vacuum and the residue was purified by preparative TLC (eluent 6.7% MeOH/DCM). The product was isolated as a white solid. ¹H NMR (400 MHz, DMSO-d₆): 7.88 (s, 1H), 7.50 (d, J=8.0 Hz, 1H), 7.43 (m, 1H), 5.18 (m, 1H), 5.08 (d, J=7.6 Hz, 1H), 4.78 (d, J=4.4 Hz, 2H), 4.26 (t, J=8.8 Hz, 1H), 3.87 (m, 3H) 2.48 (m, overlapped with DMSO-d₆, 2H); 2.25 (s, 3H). MS (m/z): 408 [M+H].

### Example 7

### Compound of Structure

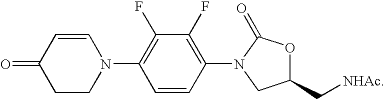

Scheme for Compound of Example 7:

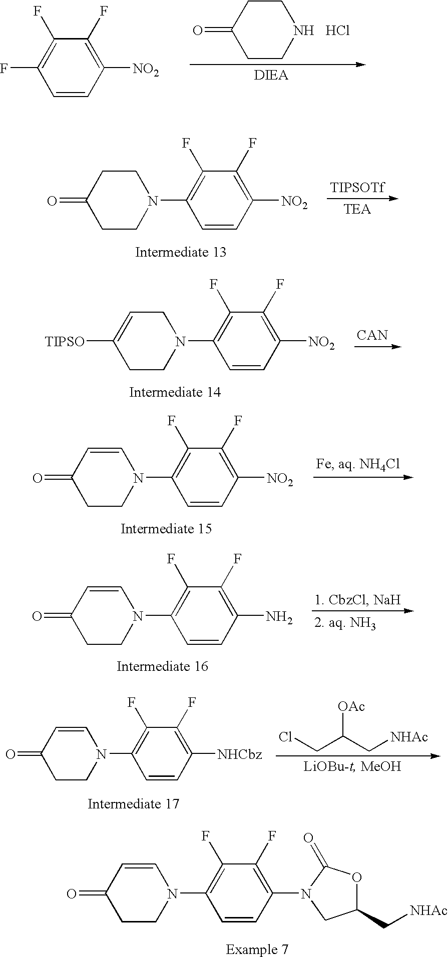

Intermediate 13. 2,3,4-Trifluoronitrobenzene (5.5 g, 30.8 mmol) was added dropwise with stirring to 4-piperidone hydrochloride (4.6 g, 33.9 mmol) and DIEA (9.2 g, 71.2 mmol) in NMP (50 mL) at ca. 0° C. The mixture was allowed to warm up to r.t. and stirred o.n. The mixture was cooled in an ice bath and quenched with ice water (ca. 300 mL). The precipitate yellow product was filtered off, washed with water and dried under vacuum. This was used for the next step without further purification.

Intermediate 14. TEA (5.3 mL, 40.7 mmol) was added to the Intermediate 13 (7.1 g, 27.7 mmol) in THF (80 mL) at 0° C., followed by triisopropylsilyl triflate (9.5 g, 32.5 mmol). The mixture was allowed to warm up to r.t. over ca. 40 min, and stirred for another 2 h. Solvent was removed on a rotary evaporator. EtOAc (120 mL) was added, and the solution washed with 10% aq. NaHCO₃ (25 mL), brine (60 mL) and dried (Na₂SO₄). Solvent was removed under vacuum and to afford the product as a red-brownish oil. This was used at the next step without purification.

Intermediate 15. CAN (17.7 g, 32.3 mmol) was added portionwise with stirring to a solution of the Intermediate 14 (11.1 g, 26.9 mmol) in dry DMF (100 mL) at 0° C. The reaction mixture was allowed to warm up to r.t. and stirred for another 4 h. Most of solvent was removed under vacuum. Water (ca. 75 mL) was added and the mixture was extracted with ethyl acetate (2×100 mL). The combined organic layers were washed with brine and dried (Na₂SO₄) Solvent was removed and the residue purified by column chromatography (gradient 20% to 30% EtOAc in petroleum ether). The product was obtained as a yellow solid (5.3 g, 78%). This was used without purification for the next step.

Intermediate 16. NH₄Cl (4.5 g, 83.3 mmol) in water (10 mL) was added to a hot solution of the Intermediate 15 (1.8 g, 7.1 mmol) in EtOH (40 mL). Iron powder (5.0 g, 89.7 mmol) was added portionwise with stirring, and the mixture at ca. 100-105° C. for 40 min. The solution was filtered through Celite, and the precipitate washed with EtOAc. EtOAc was removed under vacuum, and residue distributed between EtOAc and water. Aq. layer was washed with EtOAc (2×60 mL), and combined organic layers were washed with brine and dried (Na₂SO₄). Solvent was removed under vacuum, and the resulting product used for the next step without further purification.

Intermediate 17.60% NaH in mineral oil (0.33 g, 13.7 mmol) was added portionwise with stirring to the Intermediate 16 (1.1 g, 4.9 mmol) in THF (20 mL) at 0° C. under Ar, and the mixture was stirred at this temperature for 30 min. Benzyl chloroformate (1.25 g, 7.3 mmol) was added dropwise with stirring. The reaction mixture was allowed to warm up to r.t. and stirred o.n. The reaction was carefully quenched with water (10 mL), and THF was removed under vacuum. The residue was taken in DCM (80 mL). Organic layer was washed with brine (50 mL) and dried (Na₂SO₄). Solvent was removed under vacuum, and the residue dissolved with MeOH (20 mL). Aq. NH₃ (10 mL) was added with stirring, and the mixture was stirred at r.t. for 2 h. Solvent was removed under vacuum, and EtOAc (100 mL) was added. The organic layer was washed with brine and dried (Na₂SO₄). Solvent was removed under vacuum, and the residue was purified by column chromatography (gradient 25% to 100% DCM/petroleum ether). To afford the product as a white solid.

### Compound of Example 7

2.2M LiOBu-t in THF (0.36 mL. 0.79 mmol) was added to Intermediate 17 (70 mg, 0.20 mmol) in DMF (1.0 mL) and MeOH (0.024 mL, 0.60 mmol) at 0° C. under Ar, followed by N-[(2S)-2-acetoxy-3-chloropropyl]acetamide (193.6 mg, 1.00 mmol; prepared as described in Org. Proc. Res. Develop., 2003, p. 533). The mixture was allowed to warm up to r.t. over ca. 5 h and stirred o.n. The mixture was quenched with 10% aq. NH₄Cl and extracted with EtOAc (ca. 2×20 mL). Combined organic layers were washed with brine and dried (Na₂SO₄). Solvent was removed under vacuum and the product isolated by preparative TLC (eluent: 5% MeOH in DCM). White crystals. ¹H NMR (400 MHz): 7.30 (d, J=7.6 Hz, 1H), 7.25 (d, J=8.0 Hz, 1H), 6.96 (d, J=7.6 Hz, 1H), 5.31 (d, J=8.0 Hz, 1H), 4.84 (m, 1H), 4.09 (m, 1H), 3.97 (t, J=12.8 Hz, 2H), 3.84 (m, 1H), 3.70 (m, 1H), 2.68 (t, J=12.8 Hz, 2H). MS (m/z): 366 [M+H].

### Example 8

### Compound of Structure

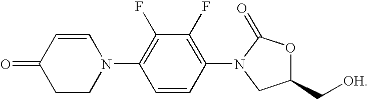

Scheme for Compound of Example 8:

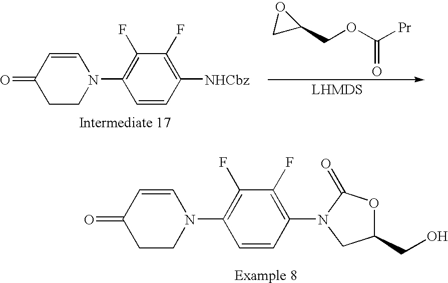

### Compound of Example 8

1.06M LHMDS (3.0 mL, 3.18 mmol) in THF was added dropwise with stirring to a solution of the Intermediate 17 (1.0 g, 2.79 mmol) in THF (8.0 mL) at −78° C., and the mixture was stirred at this temperature for 30 min. (R)-Glycidyl butyrate (0.8 mL, 5.55 mmol) was added dropwise, and the mixture was allowed to warm up to r.t. and stirred o.n. The reaction was quenched with 10% aq. NH₄Cl (15 mL), and THF was removed under vacuum. The residue was extracted with EtOAc (2×30 mL). Combined organic layers were washed with brine and dried (Na₂SO₄). Solvent was removed under vacuum. MeOH (5 mL) and 20% aqueous Cs₂CO₃ (5 mL) were added, and the mixture was stirred at r.t. for 20 min. The mixture was taken into EtOAc (50 mL), washed with water (2×15 mL), brine, and dried (Na₂SO₄). Solvent was removed under vacuum and the crude product was purified by column chromatography (2% methanol/DCM). White solid. ¹H NMR (400 MHz): 7.40 (m, 1H), 7.26 (dd, J=1.6 and 8.0 Hz, 1H), 6.97 (m, 1H), 5.33 (d, J=7.6 Hz, 1H), 4.85 (m, 1H), 4.09 (m, 1H), 4.15 (t, J=8.8 Hz, 1H), 4.06 (m, 1H), 3.99 (m, 2H), 3.82 (m, 1H), 2.70 (m, 2H), 2.15 (br. s, 1H). MS (m/z): 325 [M+H].

### Example 9

### Compound of Structure

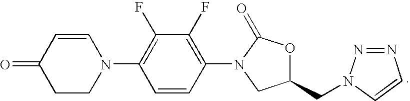

Scheme for Compound of Example 9:

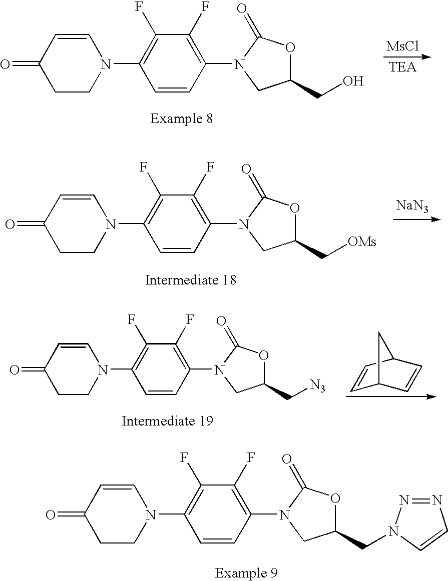

Intermediate 18. Methylsulfonyl chloride (MsCl; 79 uL, 1.00 mmol) was added dropwise with stirring to the compound of Example 8 (200 mg, 0.62 mmol) and TEA (220 mg, 2.1 mmol) in DCM (5 mL) at ca. 0° C. The mixture was stirred for 20 min and allowed to warm up to r.t. The reaction mixture distributed between water and the DCM. Aq. layer was extracted with DCM (2×10 mL), and the combined organic layers washed with brine and dried (Na₂SO₄). Solvent was removed under vacuum to afford the product that was used for the next step without purification.

Intermediate 19. A mixture of the Intermediate 18 (120 mg, 0.31 mmol) and NaN₃ (110 mg, 1.70 mmol) in DMF (5 mL) was stirred at 55° C. o.n. After cooling to r.t., water (15 mL) was added, and the reaction mixture was extracted with DCM (3×30 mL). Combined organic layers were washed with brine (30 ml) and dried (Na₂SO₄). Solvent was removed under vacuum to afford the product as a light yellow solid. This was used directly for the next step without further purification.

### Compound of Example 9

A mixture of the Intermediate 19 (80 mg, 0.3 mmol) and bicyclo[2.2.1]hepta-2,5-diene (240 mg, 2.5 mmol) in 1,4-dioxane (7 mL) under N₂ was heated at 100° C. for 3 h. Most of volatiles were removed under vacuum, and the residue was purified by column chromatography (1% MeOH/DCM). White solid. ¹H NMR (400 MHz): 7.83 (d, J=9.2 Hz 2H), 7.22 (d, J=9.2 Hz, 1H), 7.02 (m, 1H), 6.89-7.00 (m, 1H), 5.31 (d, J=8.0 Hz 1H, 5.14-5.11 (m, 1H), 4.84 (d, J=3.6 Hz, 2H), 4.26 (t, J=18.4 Hz, 1H), 3.98 (m, 3H), 2.68 (t, J=14.8 Hz, 2H). MS (m/z): 376 [M+H].

### Example 10

### Compound of Structure

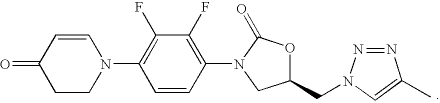

Scheme for Compound of Example 10:

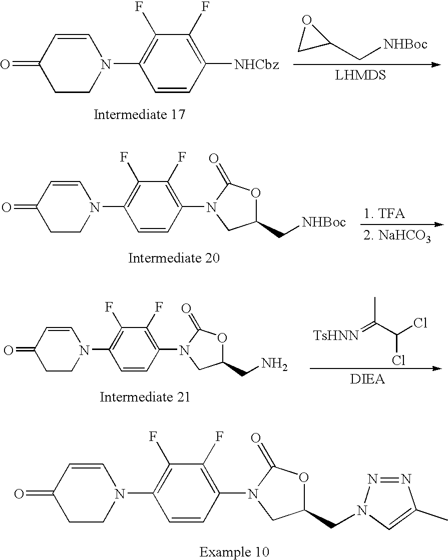

Intermediate 20. 1.06M LHMDS in THF (4.6 mL, 4.90 mmol) was added dropwise with stirring to a solution of the Intermediate 17 (700 mg, 1.96 mmol) in THF (5 mL) under N₂ at −40° C. After ca. 30 min, (S)-tert-butyl oxiran-2-ylmethylcarbamate (407 mg, 2.35 mmol) was added with stirring. The mixture was allowed to warm up to r.t. and stirred o.n. Water (5 mL) was added, and the solution extracted with EtOAc (3×8 mL). Combined organic layers were washed with brine and dried (Na₂SO₄). The product was isolated by preparative TLC (20% EtOAc/DCM) as a white solid. This was used directly for the next step.

Intermediate 21. TFA (1.0 mL) was added to the Intermediate 20 (200 mg, 0.47 mmol) in DCE (4 mL) at 0° C., and the solution was kept at r.t. for 2 h. Volatiles were removed under vacuum with a repeated addition of extra DCE (ca. 3 times). Resulted TFA salt was quenched with 5% aq. NaHCO₃ and extracted with DCM (3×10 mL). The combined organic layers were dried (Na₂SO₄), and the solvent was removed in vacuo to afford the product as an oil.

### Compound of Example 10

N′-(1,1-Dichloropropan-2-ylidene)-4-methylbenzenesulfonohydrazide (120 mg, 0.93 mmol) was added with stirring to a solution of the Intermediate 21 (100 mg, 0.31 mmol) and DIEA (150 mg, 0.45 mmol) in MeOH (4 mL) under Ar at 0° C. The reaction mixture was allowed to warm up to r.t. and stirred for 3 h. The solvent was removed under vacuum and the residue taken into DCM. Resulting mixture was washed with water and dried (Na₂SO₄). The filtrate was concentrated under vacuum and the residue was purified by preparative TLC (5% MeOH/DCM). The product was isolated as a white solid. ¹H NMR (300 MHz): 7.54 (d, J=0.6 Hz, 1H), 7.23 (dd, J=2.1 and 7.8 Hz, 1H), 7.04 (m, 1H), 6.91 (m, 1H), 5.31 (d, J=8.1 Hz, 1H), 5.12 (m, 1H), 4.74 (d, J=4.2 Hz, 2H), 4.25 (m, 1H), 3.99 (m, 3H), 2.69 (m, 2H), 2.40 (d, J=0.6 Hz, 3H). MS (m/z): 390 [M+H].

### Example 11

### Compound of Structure

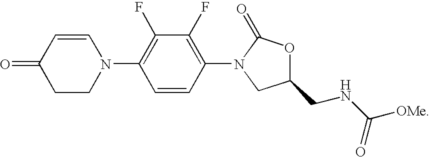

Scheme for Compound of Example 11:

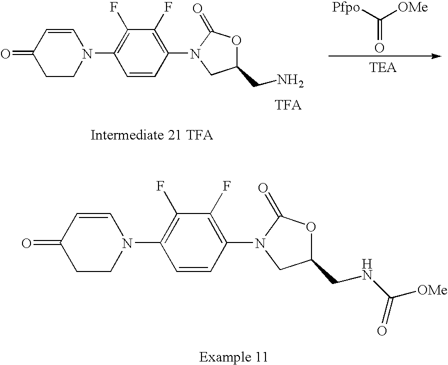

### Compound of Example 11

Pentafluorophenyl methyl carbonate (115 mg, 0.48 mmol) was added with stirring to the Intermediate 21 (TFA salt; 138 mg, 0.32 mmol) and TEA (220 μL, 1.60 mmol) in MeCN (2 mL) at ca. 0° C. The mixture was stirred at this temperature for 15 min, quenched with sat. aq. NH₄Cl solution, and extracted with EtOAc (2×10 mL). Combined organic layers were washed with brine and dried (Na₂SO₄). Solvent was removed under vacuum, and the residue purified by column chromatography (4.8% methanol/DCM) to afford the product was obtained as a white solid. ¹H NMR (400 MHz): 7.36 (t, J=7.6 Hz, 1H), 7.26 (dd, J=6.0, 2.0 Hz, 1H), 6.98 (d, J=8.4 Hz, 1H), 5.34 (d, J=7.6 Hz, 1H), 5.15 (m, 1H), 4.86 (m, 1H), 4.12 (t, J=8.8 Hz, 1H), 3.99 (t, J=7.2 Hz, 2H), 3.90 (dd, J=15.2, 6.8 Hz, 1H), 3.73 (s, 3H), 3.63 (m, 2H), 2.71 (t, J=7.6 Hz, 2H). MS (m/z): 382 [M+H].

### Example 12

### Compound of Structure

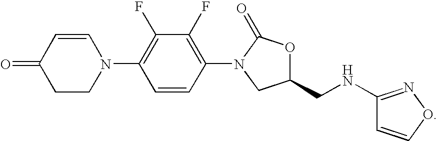

Scheme for Compound of Example 12:

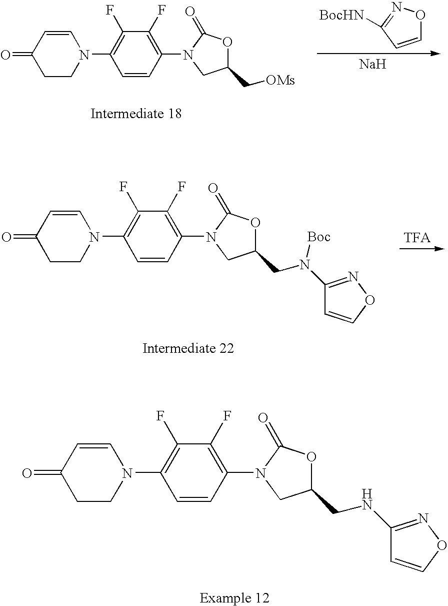

Intermediate 22. A solution of tert-butyl isoxazol-3-ylcarbamate (86 mg, 0.47 mmol) in DMF (1 mL) was added dropwise with stirring to a suspension of NaH (60% in mineral oil, 19 mg, 0.47 mmol) in DMF (1 mL). The mixture was stirred under N₂ for 15 min. at 35° C. The Intermediate 18 (0.43 mmol) in DMF (1.00 mL) was added, and the mixture was stirred at 50° C. for 1.5 h. The reaction mixture was taken into EtOAc (30 mL), washed with 10% aq. NH₄Cl (2×15 mL), brine, and dried (Na₂SO₄). Solvent was removed under vacuum and the crude product was purified by column chromatography (2% MeOH/DCM) to afford the product as a yellow solid.

### Compound of Example 12

4M HCl in ether (3 mL) was added dropwise to the solution of the Intermediate 22 (84 mg, 0.17 mmol) in DCM at 0° C., and the solution was stirred at 0° C. for 30 min, and then 1 h at r.t. Volatiles were removed under vacuum, and the residue taken into EtOAc (30 mL). The solution was washed with saturated NaHCO₃ solution (2×15 mL), brine, and dried (Na₂SO₄). Solvent was removed under vacuum and the crude product was purified by column chromatography (5% methanol/DCM). White solid. ¹H NMR (400 MHz, DMSO-d₆): 8.10 (s, 1H); 7.33 (t, J=8.5 Hz, 1H), 7.25 (d, J=8.0 Hz, 1H), 6.96 (t, J=8.1 Hz, 1H), 5.92 (s, 1H), 5.32 (d, J=7.6 Hz, 1H), 5.04 (m, 1H), 4.58 (br, 1H), 4.15 (t, J=8.8 Hz, 1H), 3.98 (t, J=7.2 Hz, 2H), 3.93 (t, J=7.6 Hz, 1H), 3.79 (dd, J=14.5, 2.9 Hz, 1H), 3.67 (dd, J=14.4, 6.4 Hz, 1H), 2.69 (t, J=7.3 Hz, 2H). MS (m/z): 391 [M+H].

### Example 13

### Compound of Structure

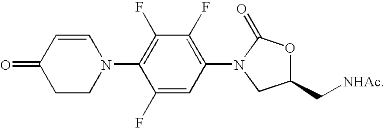

Scheme for Compound of Example 13:

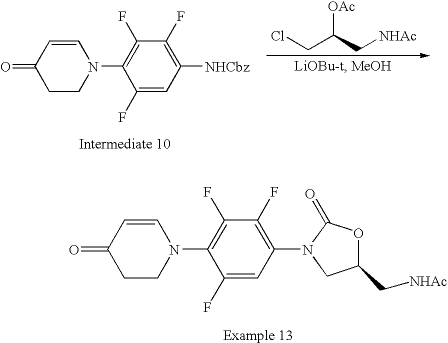

### Compound of Example 13

1M LiOBu-t in THF (0.96 mmol) was added to Intermediate 10 (90 mg, ca. 0.24 mmol) in DMF (0.18 mL) and MeOH (0.029 mL) at −10° C. under N₂, followed by N-[(2S)-2-acetoxy-3-chloropropyl]acetamide (139 mg, 0.72 mmol; prepared as described in Org. Proc. Res. Develop., 2003, p. 533). The mixture was allowed to warm up to r.t. over ca. 5 h and stirred o.n. The mixture was quenched with 10% aq. NH₄Cl (ca. 1 mL) and extracted with EtOAc (ca. 3×10 mL). Combined organic layers were washed with brine and dried (MgSO₄). Solvent was removed under vacuum and the product isolated by column chromatography (eluent: ca. 2-3% MeOH in DCM). Off-white crystals. ¹H NMR (300 MHz): 7.36-7.27 (m, 1H), 7.27-7.05 (m, 1H), 5.95 (br. t, 1H), 5.31 (d, J=8.1 Hz, 1H); 4.86 (m, 1H), 4.18-3.87 (m, 1H), 3.94-3.87 (m, 4H), 3.71 (m, 1H), 2.71 (t, J=7.5 Hz, 2H). MS (m/z): 384 [M+H].

### Example 14

### Compound of Structure

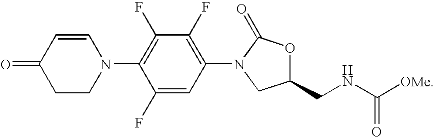

Scheme for Compound of Example 14:

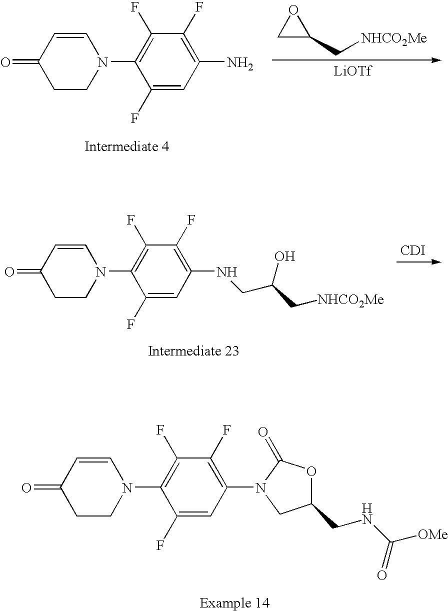

Intermediate 23. A mixture of the Intermediate 4 (500 mg, 2.1 mmol), (S)-methyl oxiran-2-ylmethylcarbamate (270 mg, 2.1 mmol), and LiOTf (970 mg, 6.2 mmol) in MeCN (4 mL) was stirred at 100° C. o.n. Solvent was removed under vacuum, and water (5 mL) was added. The mixture was extracted with EtOAc (8 mL×3), and combined organic layers dried (Na₂SO₄). Solvent was removed under vacuum, and the residue purified by preparative TLC (28% ethyl acetate/DCM) to afford the product as a light yellow solid.

### Compound of Example 14

N,N′-Carbonyldiimidazole (CDI; 0.16 g, 0.97 mmol) was added to a solution of the Intermediate 23 (181 mg, 0.48 mmol) in MeCN (2 ml), and the mixture was stirred at 80° C. under Ar o.n. Solvent was removed under vacuum, and the residue purified by preparative TLC (5% methanol/DCM). The product was obtained as a white solid. ¹H NMR (400 MHz, DMSO-d₆): 7.60 (m, 2H), 7.51 (d, J=7.6 Hz, 1H), 5.08 (d, J=7.6 Hz, 1H), 4.79 (m, 1H), 4.13 (t, J=8.8 Hz, 1H), 3.88 (m, 3H), 3.55 (s, 3H), 3.38 (overlapped with DMSO, 2H), 2.48 (overlapped with DMSO-d₆, 2H). MS (m/z): 400 [M+H].

### Example 15

### Compound of Structure

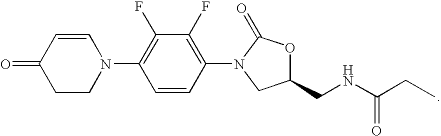

Scheme for Compound of Example 15:

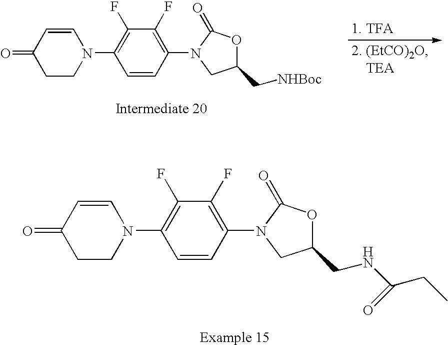

### Compound of Example 15

TFA (0.2 mL) was added to the Intermediate 20 (37 mg, 0.093 mmol) in DCM (1 mL) at 0° C. After 30 min, the solvent was removed under vacuum, and the residue was dissolved in DCM (1 mL) with TEA (64 μL, 0.47 mmol). Propionic anhydride (24 μL, 0.19 mmol) was added at 0° C., and the mixture was stirred for 30 min. The mixture was extracted with DCM (2×10 mL), and the organic layers washed with water and dried (Na₂SO₄). Solvent was removed under vacuum, and the residue was purified by TLC (10% MeOH/DCM) to afford the product as a white solid. ¹H NMR (400 MHz, DMSO-d₆): 8.22 (m, 1H), 7.60 (dd, J=7.2, 2.0 Hz, 1H), 7.39 (m, 1H), 7.26 (m, 1H), 5.08 (d, J=7.6 Hz, 1H), 4.81 (m, 1H), 4.09 (t, J=7.0 Hz, 1H), 3.95 (t, J=7.2 Hz, 2H), 3.74 (t, J=7.4 Hz, 1H), 3.45 (m, 2H), 2.53 (m, overlapped with DMSO-d₆, 2H), 2.12 (q, 4H), 0.99 (t, J=7.8 Hz, 3H). MS (m/z): 380 [M+H].

### Example 16

### Compound of Structure

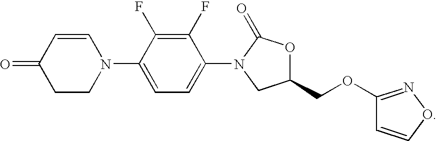

Scheme for Compound of Example 16:

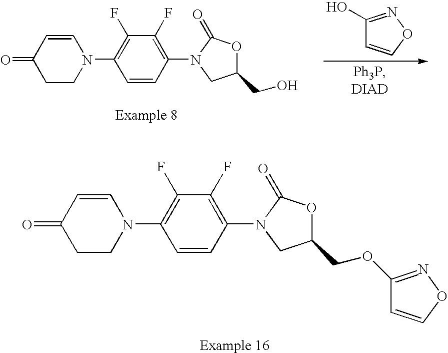

### Compound of Example 16

Diisopropyl azodicarboxylate DIAD (60 uL, 0.30 mmol) was added with stirring to PPh₃ (80 mg, 0.30 mmol) and dry THF (2 mL), and the mixture was stirred for 5 min. Isoxazol-3-ol (26 mg, 0.30 mmol) was added, after 5 min followed by the compound of Example 8 (97 mg, 0.27 mmol). The mixture was stirred for 1.5 h at r.t. Water (2 mL) was added, and the mixture was extracted with DCM (3×5 mL). Combined organic layers were washed with 0.1N HCl (3 mL), brine (3 mL) and dried (Na₂SO₄). Solvent was removed under vacuum, and the residue was purified by preparative TLC (2.4% methanol/DCM) to afford the product as a white solid. ¹H NMR (400 MHz, DMSO-d₆): 8.20 (d, J=1.6 Hz, 1H), 7.42 (t, J=7.4 Hz, 1H), 7.25 (overlapped by CHCl₃, 1H), 6.99 (t, J=8.4 Hz, 1H), 6.05 (d, J=1.6 Hz, 1H), 5.33 (d, J=3.8 Hz, 1H), 5.09 (m, 1H), 4.63 (dd, J=11.2, 3.6 Hz 1H), 4.55 (dd, J=11.6, 4.4 Hz, 1H), 4.25 (t, J=9.0 Hz, 1H), 4.03 (m, 3H), 2.70 (t, J=6.8 Hz, 2H). MS (m/z): 392 [M+H].

### Example 17

### Compound of Structure

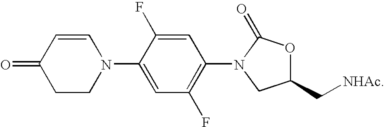

Scheme for Compound of Example 17:

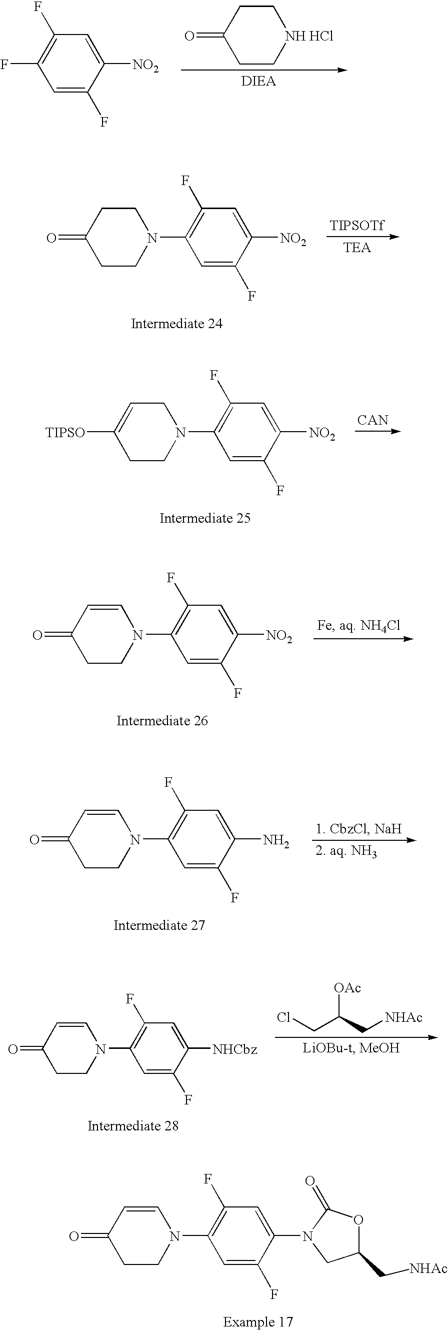

Intermediate 24. 2,4,5-Trifluoronitrobenzene (10 g, 61.2 mmol) was added dropwise with stirring to 4-piperidone hydrochloride (8.3 g, 61.2 mmol) and DIEA 18 g, 143.3 mmol) in NMP (120 mL) at ca.-5° C. under Ar. The mixture was allowed to warm up to r.t. and stirred o.n. The mixture was cooled in an ice bath and quenched with ice water (ca. 400 mL). The precipitate yellow product was filtered off, washed with water and dried under vacuum. The yellow solid obtained was used for the next step without further purification.

Intermediate 25. Triethylamine (2.3 g, 18.2 mmol) was added to the Intermediate 24 (3.5 g, 15.3 mmol) in THF (50 mL) at 0° C., followed by triisopropylsilyl triflate (5.6 g, 22.7 mmol). The mixture was allowed to warm up to r.t. over ca. 40 min, and stirred for another 2 h. Solvent was removed on a rotary evaporator. EtOAc (100 mL) was added, and the solution washed with 10% aq. NaHCO₃ (20 mL), brine (60 mL) and dried (Na₂SO₄). Solvent was removed under vacuum and to afford the product as dark oil. This was used at the next step without purification.

Intermediate 26. CAN (9.0 g, 16.4 mmol) was added portionwise with stirring to a solution of the Intermediate 25 (5.9 g, 13.2 mmol) in dry DMF (60 mL) at 0° C. The reaction mixture was allowed to warm up to r.t. and stirred for another 4 h. Most of solvent was removed under vacuum. Water was added and the mixture was extracted with EtOAc (2×100 mL). The combined organic layers were washed with brine and dried (Na₂SO₄). Solvent was removed and the residue purified by column chromatography (gradient 20% to 30% EtOAc in petroleum ether). The product was obtained as a yellow solid.

Intermediate 27. NH₄Cl (4.8 g, 89.7 mmol) in water (20 mL) was added to a hot solution of the Intermediate 26 (2.1 g, 8.2 mmol) in EtOH (60 mL). Iron powder (5.2 g, 92.8 mmol) was added portionwise with stirring, and the mixture at ca. 100-105° C. for 40 min. The solution was filtered through Celite, and the precipitate washed with EtOH (5×10 mL). EtOH was removed under vacuum, and residue distributed between EtOAc (ca. 50 mL) and water (10 mL). Aq. layer was washed with EtOAc (2×60 mL), and combined organic layers were washed with water (3×7 mL), brine, and dried (MgSO₄). Solvent was removed under vacuum to afford the product as yellow crystals. Yield 1.5 g (81%).

Intermediate 28. 2M aq. LiOH (0.53 mL, 1.06 mmol) was chilled to ca. 5° C. and then added with stirring to the Intermediate 27 (138 mg, 0.53 mmol) in THF (3 mL) at 0° C., followed by benzyl chloroformate (0.093 mL, 0.64 mmol) in THF (0.25 mL). The mixture was stirred and allowed to warm up to r.t. over ca. 5 h. THF was removed under vacuum, and the mixture was extracted with EtOAc (3×20 mL). Combined organic layers were washed with 10% aq. citric acid (ca. 7×20 mL), water (3×15 mL), brine, and dried (MgSO₄). Solvent was removed under vacuum, and the crude was crystallized from ether and dried under vacuum. White solid.

### Compound of Example 17

1M LiOBu-t in THF (0.84 mL, 0.84 mmol) was added to Intermediate 28 (72 mg, 0.21 mmol) in DMF (0.12 mL) and MeOH (0.026 mL) at −10° C. under nitrogen, followed by N-[(2S)-2-acetoxy-3-chloropropyl]acetamide (122 mg, 0.63 mmol; prepared as described in Org. Proc. Res. Develop., 2003, p. 533). The mixture was allowed to warm up to r.t. over ca. 5 h and stirred o.n. The mixture was quenched with 10% aq. NH₄Cl (1.5 mL) and extracted with EtOAc (3×15 mL). Combined organic layers were washed with brine and dried (MgSO₄). Solvent was removed under vacuum and the product isolated by column chromatography (5% MeOH in DCM). Off-white crystals. ¹H NMR (300 MHz): 7.44 (m, 1H), 7.23 (dd, J=2.1 and 7.8 Hz, 1H), 6.96 (m, 1H), 5.96 (br. t, 1H), 5.31 (d, J=7.8 Hz, 1H); 4.84 (m, 1H), 4.11 (m, 1H), 3.96 (m, 2H), 3.83 (m, 1H), 3.80-3.62 (m, 2H), 2.71 (t, J=7.5 Hz, 2H), 2.07 (s, 3H). MS (m/z): 366 [M+H].

### Example 18

### Compound of Structure

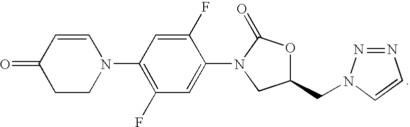

Scheme for Compound of Example 18:

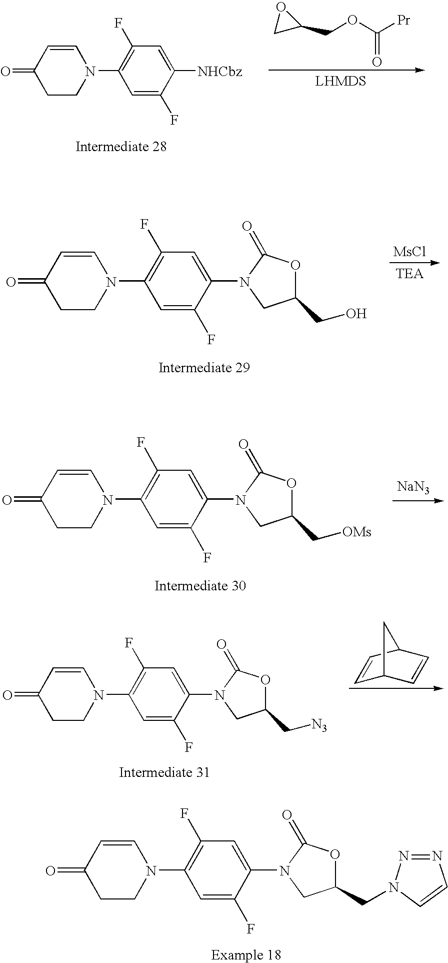

Intermediate 29. 1.06M LHMDS in THF (1.5 mL, 1.09 mmol) was added dropwise with stirring to a solution of the Intermediate 28 (0.6 g, 1.68 mmol) in THF (8.0 mL) at −78° C., and the mixture was stirred at this temperature for 30 min. (R)-Glycidyl butyrate (0.4 mL, 2.28 mmol) was added dropwise, and the mixture was allowed to warm up to r.t. and stirred o.n. The reaction was quenched with 10% aq. NH₄Cl (15 mL), and THF was removed under vacuum. The residue was extracted with EtOAc (2×30 mL). Combined organic layers were washed with brine and dried (Na₂SO₄). Solvent was removed under vacuum. MeOH (5 mL) and 20% aqueous Cs₂CO₃ (5 mL) were added, and the mixture was stirred at r.t. for 20 min. The mixture was taken into EtOAc (50 mL), washed with water (2×15 mL), brine, and dried (Na₂SO₄). Solvent was removed under vacuum and the crude product was purified by column chromatography (2% methanol/DCM). The product isolated as a white solid.

Intermediate 30. MsCl (350 mg, 2.1 mmol) was added dropwise with stirring to the Intermediate 29 (280 mg, 0.91 mmol) and TEA (320 mg, 3.1 mmol) in DCM (5 mL) at ca. 0° C. The mixture was stirred for 20 min and allowed to warm up to r.t. The reaction mixture distributed between water and the DCM. Aq. layer was extracted with DCM (2×10 mL), and the combined organic layers washed with brine and dried (Na₂SO₄). Solvent was removed under vacuum to afford the product that was used for the next step without purification.

Intermediate 31. A mixture of the Intermediate 30 (350 mg, 0.91 mmol) and NaN₃ (296 mg, 4.56 mmol) in DMF (6 mL) was stirred at 55° C. o.n. After cooling to r.t., water (15 mL) was added, and the reaction mixture was extracted with DCM (3×30 mL). Combined organic layers were washed with brine (30 ml) and dried (Na₂SO₄). Solvent was removed under vacuum to afford the product as a light yellow solid. This was used directly for the next step without further purification.

### Compound of Example 18

A mixture of the Intermediate 31 (220 mg, 0.6 mmol) and bicyclo[2.2.1]hepta-2,5-diene (600 mg, 6.2 mmol) in 1,4-dioxane (15 mL) under N₂ was heated at 100° C. for 10 h. Most of volatiles were removed under vacuum, and the product was purified by column chromatography (1% MeOH/DCM). White solid. ¹H NMR (400 MHz): 7.83 (d, J=9.2 Hz, 2H), 7.13 (m, 2H), 6.89 (m, 1H), 5.31 (d, J=7.8 Hz, 1H), 5.13 (m, 1H), 4.83 (d, J=7.2 Hz, 2H), 4.27 (t, J=8.4 Hz, 1H), 3.95 (m, 1H), 3.94 (t, J=8.6 Hz, 2H), 2.68 (t, J=8.6 Hz, 2H). MS (m/z): 376 [M+H].

### Example 19

### Compound of Structure

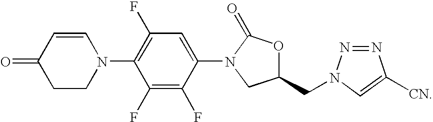

Scheme for Compound of Example 19:

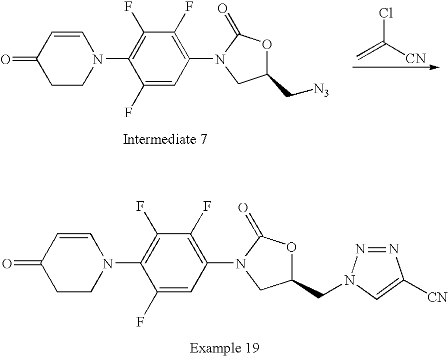

### Compound of Example 19

2-Chloroacrylonitrile (44 uL, 0.54 mmol) was added to the Intermediate 7 (100 mg, 0.27 mmol) in DMF (1 mL) under Ar. The reaction mixture was stirred at 95° C. for 2 d. After cooling to r.t., the mixture was taken into water (5 mL), extracted with EtOAc (3×5 mL), and dried (Na₂SO₄). The product was purified by preparative TLC (5% methanol/DCM). Light yellow solid. ¹H NMR (400 MHz): 8.28 (s, 1H), 7.20 (m, 1H), 7.09 (d, J=7.6 Hz, 1H), 5.32 (d, J=7.6 Hz, 1H), 5.18 (m, 1H), 4.94 (dd, J=14.4, 3.2 Hz, 1H), 4.86 (dd, J=15.2, 5.2 Hz, 1H), 4.35 (t, J=8.8 Hz, 1H), 4.06 (dd, J=9.2, 6.4 Hz, 1H), 3.92 (t, J=7.4 Hz, 2H), 2.70 (t, J=7.2 Hz, 2H). MS (m/z): 419 [M+H].

### Example 20

### Compound of Structure

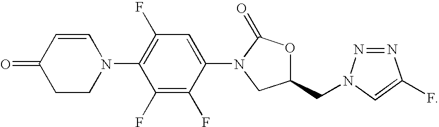

Scheme for Compound of Example 20:

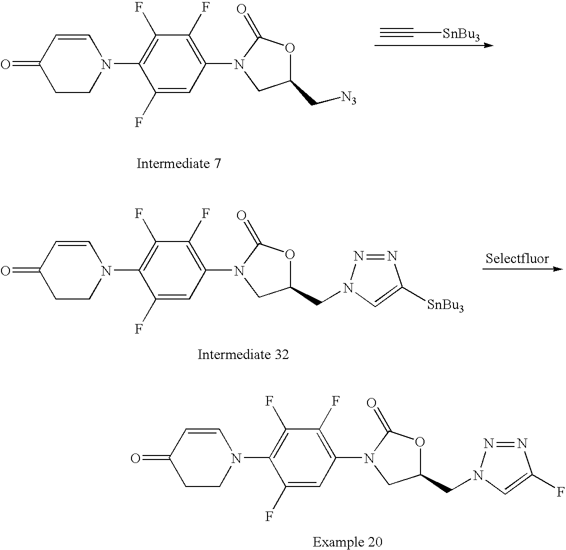

Intermediate 32. Tributylethynylstannane (260 μL, 0.90 mmol) was added to the Intermediate 7 in toluene (6 mL), and the mixture was stirred at 70° C. for 2 d. Solvent was removed under vacuum, and the residue was purified by column chromatography (2.4% methanol/DCM) to afford the product. MS (m/z): 684 [M+H].

### Compound of Example 20

1-Chloromethyl-4-fluoro-1,4-diazoniobicyclo[2.2.2]octane bis(tetrafluoroborate) (Selectfluor™; 278 mg, 0.78 mmol) was added to the Intermediate 32 (447 mg, 0.65 mmol) in MeCN (6 mL). The reaction mixture was stirred for 3 d at r.t., quenched with brine, and extracted with DCM (2×10 mL). Combined organic layers were dried (Na₂SO₄), and concentrated under vacuum. The residue was purified by preparative TLC (2.4% methanol/DCM) to afford the product as a pale yellow solid. ¹H NMR (400 MHz): 8.19 (s, 1H), 7.97 (d, J=10.0 Hz, 1H), 7.80 (s, 1H), 7.43 (ddd, J=12.0, 6.8, 2.4 Hz, 1H), 5.22 (m, 1H), 4.87 (d, J=4.8 Hz, 2H), 4.27 (t, J=8.8 Hz, 1H), 3.92 (dd, J=8.8, 5.6 Hz, 1H), 3.85 (t, J=7.2 Hz, 2H), 2.61 (td, J=8.0, 2.8 Hz, 2H). MS (m/z): 412 [M+H].

### Example 21

### Compound of Structure

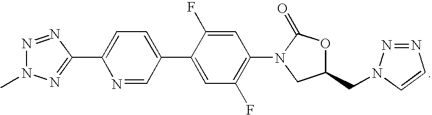

Scheme for Compound of Example 21:

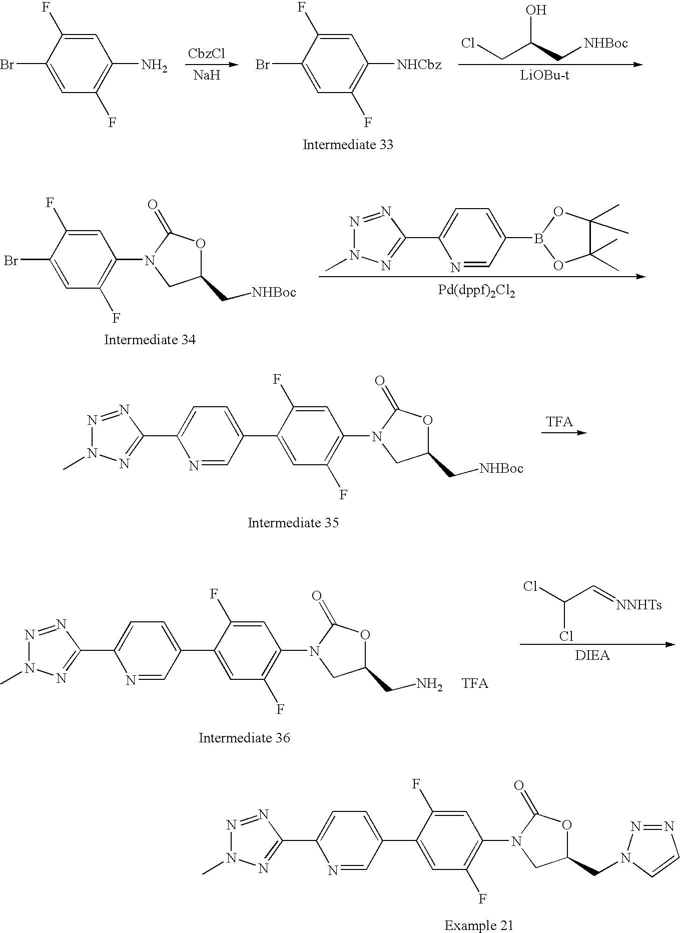

Intermediate 33. To a solution of 4-bromo-2,5-difluoroaniline (1.7 g, 8.2 mmol) in dry THF (25 mL) was added NaH (60% dispersion in mineral oil, 1.0 g, 25.1 mmol) in portions, and the mixture was cooled to 0° C. Benzyl chloroformate (9.0 mmol) was added dropwise, and the mixture was stirred for 16 h at r.t. Water (5 mL) was added, and THF removed under vacuum. Methanol (25 mL) and conc. aq. ammonia (ca. 5 mL) were added, and the solution was stirred for 1 h at r.t. The solution was concentrated under vacuum and extracted with EtOAc (3×20 mL). Combined organic layers were dried (Na₂SO₄), solvent was removed under vacuum, and the product was purified by column chromatography (5% ethyl acetate/petroleum ether). White solid.

Intermediate 34. (S)-tert-butyl 3-chloro-2-hydroxypropylcarbamate (122 mg, 0.58 mmol; prepared as described in Org. Proc. Res. Develop., 2003, p. 533) was added to the Intermediate 33 (100 mg, 0.29 mmol) in MeCN (0.5 mL) at 0° C., followed by t-BuOLi (2.2 M in THF, 0.33 mL, 0.73 mmol). The reaction mixture was stirred at 0° C. for 3 h and then o.n. at r.t. Water (5 mL) was added and the mixture was extracted with ethyl acetate (3×15 mL). Combined organic layers were dried (Na₂SO₄), solvent was removed under vacuum, and the product was purified by preparative TLC (5% methanol/DCM). The product was obtained as light yellow oil.

Intermediate 35. 2-(2-Methyl-2H-tetrazol-5-yl)-5-(4,4,5,5-tetramethyl-1,3,2-dioxaborolan-2-yl)pyridine (230 mg, 0.8 mmol) and Intermediate 34 (320 mg, 0.78 mmol) were dissolved in DMF (15 mL) under Ar. KOAc (230 mg, 2.4 mmol) and PdCl₂(dppf)DCM (58 mg, 0.078 mmol) were added, the mixture was degassed, and then stirred at 80° C. o.n. Resulted solution was filtered through Celite and washed with 50 mL of EtOAc. The filtrate was concentrated, washed with 10% NH₄Cl, brine, and dried (Na₂SO₄). Solvent was removed under vacuum, and the product was purified by preparative TLC (5% methanol/DCM). White solid.

Intermediate 36. TFA (0.75 mL) was added with stirring to the Intermediate 35 (23 mg, 0.047 mmol) in DCE (2.5 mL) at 0° C. The reaction mixture was stirred for 2 h at r.t. and concentrated under vacuum to afford the product that was used directly at the next step.

### Compound of Example 21

N′-(2,2-Dichloroethylidene)-4-methylbenzenesulfonohydrazide (42 mg, 0.11 mmol; prepared as described in Heterocycles, 1998, p. 895) was added with stirring to the Intermediate 36 (50 mg, 0.10 mmol) and DIEA (55 mg, 0.17 mmol) in MeOH (4 mL) at 0° C. The reaction mixture was stirred at 0° C. for 3 h, and then concentrated under vacuum. Water (ca. 5 mL) was added, and the mixture was extracted with dichloroethane (3×15 ml). Combined organic layers were dried (Na₂SO₄), concentrated under vacuum, and the residue was purified by preparative TLC (5% DCM/methanol). The product was obtained as a white solid. ¹H NMR (400 MHz): 8.89 (s, 1H), 8.48 (d, J=7.6 Hz, 1H), 8.06 (d, J=7.8 Hz, 1H), 7.85 (d, J=7.6 Hz, 2H), 7.29 (m, 2H), 5.18 (m, 1H), 4.86 (d, J=7.6 Hz, 2H), 4.58 (s, 3H), 4.38 (t, J=8.8 Hz, 1H), 4.09-4.12 (m, 1H). MS (m/z): 440 [M+H].

### Example 22

### Compound of Structure

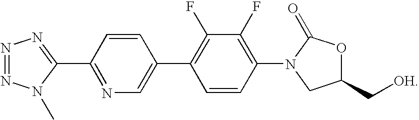

Scheme for Compound of Example 22:

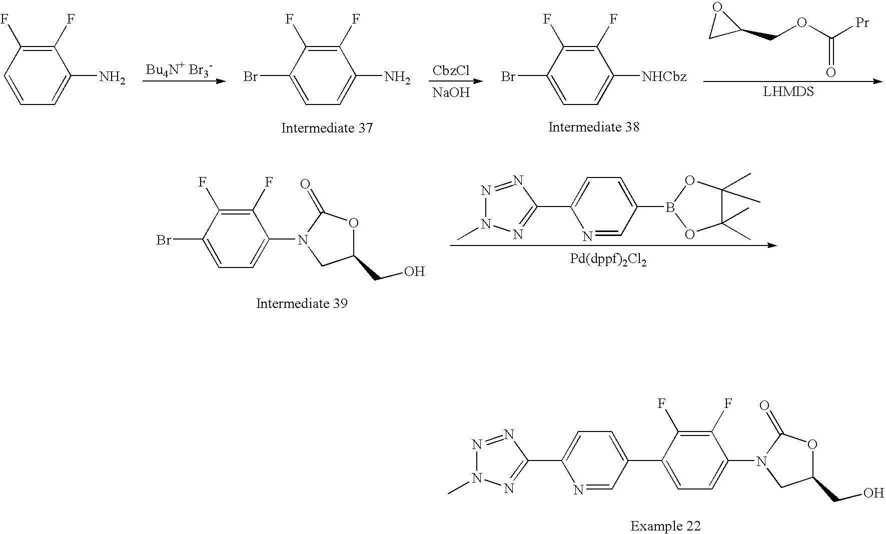

Intermediate 37. Bu₄NBr₃ (2.9 g, 6.0 mmol) in DCM (10 mL) was added dropwise with stirring to 2,3-difluoroaniline (645 mg, 5.0 mmol) in DCM (10 mL). The reaction was stirred at r.t. until the starting materials disappeared. Solvent was then removed under vacuum, water added, and the mixture was extracted with EtOAc (2×60 mL). Combined organic layers were washed with brine, dried (Na₂SO₄), and concentrated under vacuum to afford the product was obtained as a colorless oil. MS (m/z): 209 [M+H].

Intermediate 38. Benzyl chloroformate (1.1 mL, 7.5 mmol) was added dropwise with stirring to the Intermediate 37 (1.0 g, 4.8 mmol) in 10% aq. NaOH (15 mL)) and THF (30 mL) at ca. 0° C. The reaction mixture was stirred at r.t. for ca. 6 h. The reaction was quenched with 10% NH₄Cl solution and extracted with DCM (2×50 mL). Combined organic layers were washed with brine, dried (Na₂SO₄), and concentrated under vacuum. The residue was purified by preparative TLC (10% ethyl acetate/petroleum ether) to give the product as a white solid. ¹H NMR (400 MHz, CDCl₃): 7.88 (m, 1H); 7.40 (m, 5H); 6.90 (m, 1H); 5.25 (s, 2H).

Intermediate 39. 1.06M Lithium hexamethyldisilylamide in THF (LHMDS; 1.2 mL, 1.3 mmol) was added dropwise with stirring to a solution of the Intermediate 38 (350 mg, 1.0 mmol) in THF (8.0 mL) at −78° C., and the mixture was stirred at this temperature for 30 min. (R)-Glycidyl butyrate (290 mg, 2.0 mmol) was added dropwise, and the mixture was allowed to warm up to r.t. and stirred o.n. The reaction was quenched with 10% aq. NH₄Cl (15 mL), and THF was removed under vacuum. The residue was extracted with EtOAc (2×20 mL). Combined organic layers were washed with brine and dried (Na₂SO₄). Solvent was removed under vacuum and the crude product was purified by preparative TLC (10-20% methanol/DCM) to give the product as a white solid. ¹H NMR (400 MHz): 7.30 (m, 2H), 4.81 (m, 1H), 4.11 (t, J=8.8 Hz, 1H), 4.01 (m, 2H), 3.78 (m, 1H).

### Compound of Example 22

5-Bromo-2-(1-methyl-1H-tetrazol-5-yl)pyridine (2.44 g, 10 mmol) was dissolved in 30 mL of anhydrous DMSO. To this solution was added bis-(pinocalato)diboron (5.08 g, 20 mmol), followed by KOAc (4.00 g, 40 mmol) and PdCl₂(dppf)DCM (0.75 g, 1 mmol). The reaction mixture was degassed, and then stirred at 80° C. o.n. Resulted solution was filtered through Celite, and the precipitate was washed with EtOAc (100 mL). The filtrate was concentrated and washed with 10% NH₄Cl, brine, and dried (Na₂SO₄). Solvent was removed under vacuum, and the residue was dissolved in ether and filtered through a short silica gel pad. The filtrate was concentrated and the formed solid was washed with methanol. Thus isolated [2-(1-methyl-1H-tetrazol-5-yl)pyridyl-5-yl)(pinacolato)boron was obtained as a white solid [¹H NMR (400 MHz): 9.10 (s, 1H); 8.25 (s, 2H); 4.48 (s, 3H); 1.48 (s, 12H)]. This compound (68 mg, 0.24 mmol) wad added to the Intermediate 39 (50 mg, 0.16 mmol) in dioxane (5 mL) and water (1 mL), followed by PdCl₂(dppf)DCM (18 mg, 0.024 mmol) and K₂CO₃ (88 mg, 0.64 mmol). The reaction mixture was degassed, and then stirred at 80° C. o.n. The reaction mixture was filtered through Celite, and the precipitate was washed with EtOAc (50 mL). The filtrate was concentrated and washed with 10% NH₄Cl, brine, and dried (Na₂SO₄). Solvent was removed under vacuum, and the residue was purified by preparative TLC (5% methanol/DCM), to afford the product was obtained as a white solid. ¹H NMR (400 MHz): 8.96 (m, 1H), 8.36 (d, J=8.0 Hz 1H), 8.05 (d, J=8.0 Hz, 1H), 7.54-7.60 (m, 1H), 7.32 (m, 1H), 4.88 (m, 1H), 4.51 (s, 3H), 4.18 (dd, J=8.0 Hz, 1H), 4.05 (m, 2H), 3.82 (dd, J=3.6, 9.2 Hz, 1H). MS (m/z): 389 [M+H].

### Example 23

### Compound of Structure

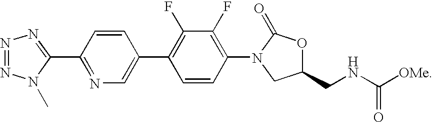

Scheme for Compound of Example 23:

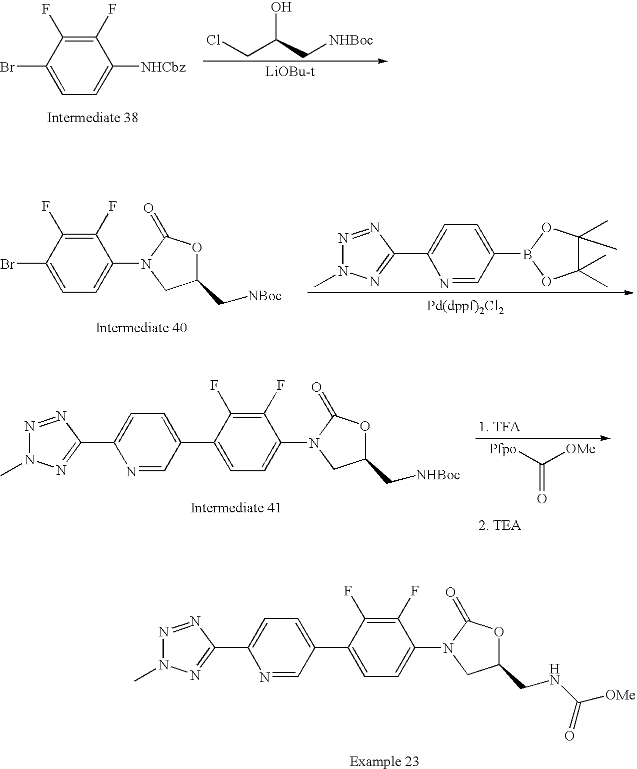

Intermediate 40. (S)-tert-butyl 3-chloro-2-hydroxypropylcarbamate (120 mg, 0.57 mmol; prepared as described in Org. Proc. Res. Develop., 2003, p. 533) was added to the Intermediate 38 (150 mg, 0.44 mmol) in DMF (0.5 mL) at ca.-10° C., followed by t-BuOLi (2.2 M, 480 μL, 1.06 mmol). The reaction mixture was stirred at 0° C. for 3 h and then o.n. at r.t. Saturated aq. NH₄Cl (ca. 5 mL) was added, and the mixture was extracted with EtOAc (3×15 mL). Combined organic layers were dried (Na₂SO₄), solvent was removed under vacuum, and the product was purified by preparative TLC (5% methanol/DCM). The desired product was obtained as a colorless solid. MS (m/z): 429 [M+Na].

Intermediate 41. The compound was prepared by the coupling procedure described for Compound of Example 22, except that [2-(1-methyl-1H-tetrazol-5-yl)pyridyl-5-yl)(pinacolato)boron (40 mg, 0.14 mmol) was reacted with above Intermediate 40 (57 mg, 0.14 mmol) instead of the Intermediate 39. White solid. MS (m/z): 488 [M+H].

### Compound of Example 23

TFA (0.4 mL) was added to the Intermediate 41 (25 mg, 0.051 mmol) in DCE (2 mL) at 0° C., and the mixture was stirred for 1 h at 0° C. Solvent removed under vacuum, and the residue taken into MeCN (2 mL) with TEA (36 μL). Pentafluorophenyl methyl carbonate (19 mg) was added, and the mixture was stirred for 30 min at r.t. Solvent was removed under vacuum, and the residue was purified by preparative TLC (5% methanol/DCM) to afford the product as a white solid (18 mg, 78%). ¹H NMR (400 MHz): 8.99 (s, 1H), 8.28 (s, 2H), 7.59 (m, 3H), 4.81 (m, 1H), 4.50 (s, 3H), 4.20 (t, J=8.8 Hz, 1H), 3.89 (t, J=6.8 Hz, 1H), 3.57 (s, 3H), 3.41 (t, J=5.6 Hz, 2H). MS (m/z): 446.0 [M+H].

### Example 24

### Compound of Structure

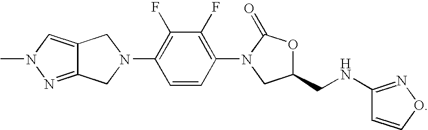

Scheme for Compound of Example 24:

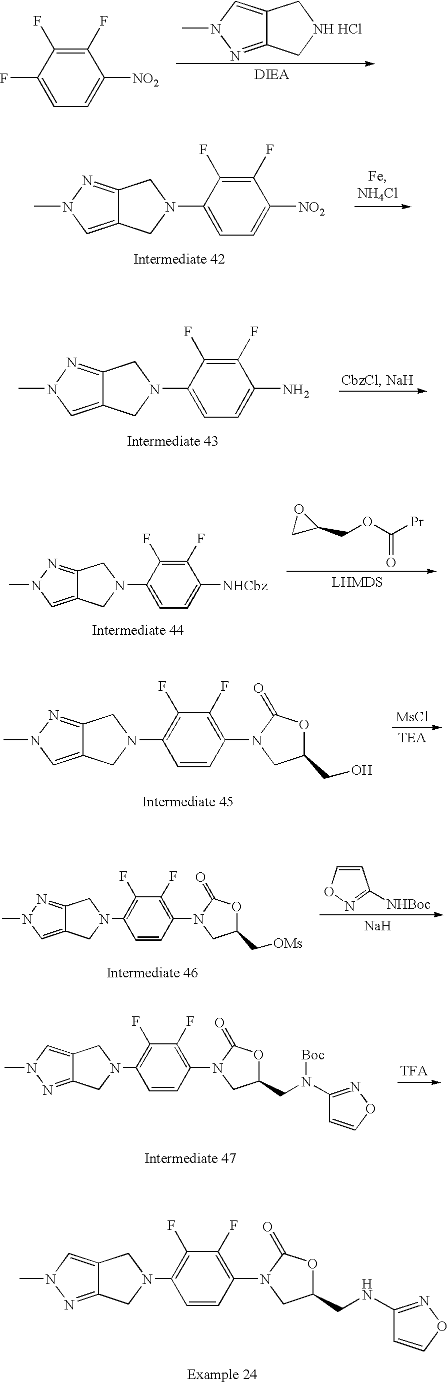

Intermediate 42. DIEA (3.8 mL) was added dropwise with stirring to 2-methyl-2,4,5,6-tetrahydropyrrolo[3,4-c]pyrazole hydrochlororide (1.0 g, 7.04 mmol; prepared as described in JP 6073056) and 2,3,4-trifluoronitrobenzene (1.5 g, 8.45 mmol) in MeCN (100 mL) at −10° C. The mixture was allowed to warm up to r.t. and stirred for 6 h. Solvent was removed under vacuum, and the residue was taken into EtOAc (60 mL), washed with water (40 mL×3), brine (40 mL), and dried (Na₂SO₄). Solvent was removed under vacuum, and the product was purified by column chromatography (gradient 17% to 75% petroleum ether/ethyl acetate). Yellow solid. ¹H NMR (400 MHz): 7.54 (m, 1H), 7.27 (d, J=6.4 Hz, 1H), 6.95 (m, 1H), 4.54 (s, 2H), 4.49 (s, 2H), 3.85 (s, 3H).

Intermediate 43. NH₄Cl (1.14 g, 21.3 mmol) in water (3 mL) was added to a hot solution of the Intermediate 42 (0.60 g, 2.1 mmol) in EtOH (6 mL). Iron powder (5.2 g, 92.8 mmol) was added portionwise with stirring, and the mixture at 95° C. for 1 h. The solution was filtered through Celite, and the precipitate washed with EtOH. EtOH was removed under vacuum, and residue distributed between EtOAc (20 mL) and water (10 mL). Aq. layer was washed with EtOAc, and combined organic layers were washed with water (3×7 mL), brine, and dried (MgSO₄). Solvent was removed under vacuum to afford the product as yellow crystals. ¹H NMR (400 MHz): 7.27 (d, J=2.8 Hz, 1H), 6.90 (m, 1H), 6.45 (m, 1H), 4.35 (d, J=2.0 Hz, 2H), 4.28 (s, 2H), 3.85 (s, 3H), 1.64 (s, 2H). MS (m/z): 251 [M+H].

Intermediate 44. 60% NaH in mineral oil (224.6 mg, 5.62 mmol) was added portionwise with stirring to the Intermediate 43 (391.6 mg, 1.56 mmol) in THF (6 mL) at −10° C., followed by a solution of benzyl chloroformate (0.4 mL, 2.82 mmol) in THF (2 mL). The mixture was allowed to warm up to r.t. and stirred o.n. The reaction was quenched with saturated NH₄Cl (5 mL) and extracted with EtOAc (3×20 mL). Combined organic layers were washed with brine (15 mL), and dried (Na₂SO₄). Solvent was removed under vacuum, and the residue was purified by column chromatography (80% to 75% petroleum ether/ethyl acetate) to afford the product as a white solid. ¹H NMR (400 MHz): 8.12 (s, 1H), 7.99 (s, 1H), 6.10 (t, J=15.6 Hz, 6H), 7.09 (m, 1H), 5.21 (s, 1H), 5.13 (s, 1H), 4.31 (s, 2H), 4.24 (s, 2H). MS (m/z): 385 [M+H].

Intermediate 45. 1.06M LHMDS in THF (0.19 ml, 0.20 mmol) was added dropwise with stirring to a solution of the Intermediate 44 (65.0 mg, 0.17 mmol) in THF (2.0 mL) at −78° C., and the mixture was stirred at this temperature for 1 h. (R)-Glycidyl butyrate (48.7 mg, 0.34 mmol) was added dropwise, and the mixture was allowed to warm up to r.t. and stirred o.n. The reaction was quenched with saturated aq. NH₄Cl (10 mL), and extracted with EtOAc (3×15 mL). Combined organic layers were washed with brine and dried (Na₂SO₄). Solvent was removed under vacuum and the crude product was purified by preparative TLC (5% DCM/MeOH) gave the desired product as a white solid. ¹H NMR (400 MHz): 7.25 (d, J=14.4 Hz, 1H), 7.15 (t, J=14.4 Hz, 1H), 7.04 (m, 1H), 4.77 (t, J=14.4 Hz, 1H), 4.47 (s, 1H), 4.40 (s, 1H), 3.99 (t, J=16.8 Hz, 2H) 3.90 (t, J=14.8 Hz, 1H), 3.81 (s, 3H), 3.71 (t, J=6.1 Hz, 2H). MS (m/z): 351 [M+H].

Intermediate 46

MsCl (20 μL, 0.27 mmol) was added dropwise with stirring to the Intermediate 45 (78.9 mg, 0.22 mmol) and TEA (94 μL, 0.67 mmol) in DCM (2 mL) at ca. 0° C. The mixture was stirred for 30 min and allowed to warm up to r.t. The reaction mixture distributed between water (5 mL) and DCM (10 mL). Aq. layer was extracted with DCM (2×10 mL), and the combined organic layers washed with brine and dried (Na₂SO₄). Solvent was removed under vacuum to afford the product as a white solid.

Intermediate 47. A solution of tent-butyl isoxazol-3-ylcarbamate (45.0 mg, 0.24 mmol) in DMF (1 mL) was added dropwise with stirring to a suspension of NaH (60% in mineral oil, 9.8 mg, 0.24 mmol) in DMF (2 mL). The mixture was stirred under Ar for 15 min. at 35° C., and then cooled down to r.t. The Intermediate 46 (95.1 mg, 0.22 mmol) in DMF (1 mL) was added, and the mixture was stirred at 50° C. for 1.5 h. The reaction mixture was taken into EtOAc (30 mL), washed with 10% aq. NH₄Cl (2×15 mL), brine, and dried (Na₂SO₄). Solvent was removed under vacuum and the crude product was purified by preparative TLC (2.4% MeOH/DCM) to afford the product as a white solid. MS (m/z): 517 [M+H].

### Compound of Example 24

TFA (0.2 mL) was added dropwise to the solution of the Intermediate 47 (25 mg, 0.048 mmol) in DCE (1 mL) at 0° C., and the solution was stirred at 0° C. for 1 h. The reaction was quenched with 5% aq. NaHCO₃ (5 mL) and extracted with DCM (3×3 mL), brine, and dried (Na₂SO₄). Solvent was removed under vacuum and the crude product was purified by preparative TLC (5% MeOH/DCM) to afford the product as a white solid. ¹H NMR (400 MHz): 8.03 (s, 1H), 7.27 (s, 1H), 7.09 (m, 1H), 5.75 (s, 1H), 4.97 (t, J=6.4 Hz, 1H), 4.39 (d, J=12.8 Hz, 2H), 4.32 (s, 2H), 4.03 (t, J=17.6 Hz, 1H), 3.85 (s, 3H), 3.82 (d, J=8.8 Hz, 1H), 3.70 (d, J=14.4 Hz, 1H), 3.56 (m, 1H). MS (m/z): 417 [M+H].

### Example 25

### Preparation of a Form a Crystal of the Compound of Example 3

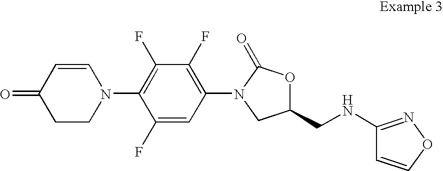

A Form A crystal of the compound of Example 3 was obtained using the procedures described below in Methods A through F.

Method A. The compound of Example 3 (200 mg) in EtOH (ca. 8 mL) was agitated at 80° C. After ca. 30 min, the compound was completely dissolved. An extra ca. 120 mg of the compound was added in 3 portions (2×50 mg, and then ca. 20 mg), allowing dissolution of each preceding portion. Extra EtOH (ca. 0.25 mL) was added and the mixture was agitated for another 30 min. The resulting nearly homogenous solution was allowed to cool down to r.t. Supernatant was removed, and the crystals of the compound of Example 3 were dried at 60° C. under vacuum. Yield 240 mg (75%). HPLC: Rₜ 13.8 min.

Method B. The compound of Example 3 (200 mg) in EtOH (ca. 4 mL) was agitated at 80° C. After ca. 30 min, extra EtOH (ca. 0.5 mL) was added, and the mixture was agitated for another 30 min. When the compound was completely dissolved, water (ca. 1 mL) was added. The solution was then left at r. t. overnight. Part of the solvent was removed under reduced pressure until precipitation started (by weight, ca. 2.3 g of solvent was evaporated). The suspension was heated to reflux, and the solution was rendered homogenous. The solution was left to crystallize at r.t. The precipitate was filtered, and the crystals of the compound of Example 3 were dried at 60° C. under vacuum. Yield 148 mg (74%).

Method C. The compound of Example 3 (200 mg) in EtOH-methyl tert-butyl ether (MTBE) 2:1 (ca. 5 mL) was agitated at 80° C. Additional solvent (1 mL) was added over ca. 3 h, followed by extra compound (ca. 25 mg), and then extra solvent (ca. 0.5 mL). The solution was cooled down to r.t. Supernatant was removed, and the crystals of the compound of Example 3 were dried at 60° C. under vacuum. Yield 178 mg (65%).

Method D. The compound of Example 3 (200 mg) in EtOH-EtOAc 1:1 (10 mL) was agitated at 80° C. Extra compound (ca. 5×25 mg) was added, allowing dissolution of each preceding portion. Hexane (11 mL) was added, followed by extra EtOH-EtOAc 1:1 (1 mL). The solution was heated until it became clear, and then cooled down to r.t. to obtain the crystals of the compound of Example 3.

Method E. A solution of the compound of Example 3 in EtOAc-hexane was concentrated to obtain the crystals of the compound of Example 3.

Method F. The compound of Example 3 was crystallized as described above for Method A in EtOH—H₂O 3:2 to obtain the crystals of the compound of Example 3.

Crystals of the compound of Example 3 obtained from the above methods A through F were analyzed using the techniques of ¹H NMR spectroscopy, elemental analysis, high resolution mass spectrometry (HRMS), X-ray power diffraction (XRPD) spectroscopy using Cu Kα radiation, infrared (IR) and ultraviolet (UV) spectroscopy, and differential scanning calorimetry (DSC). Crystals obtained from the each of above methods A through F exhibited substantially identical spectra, summarized below. This suggests that a single polymorph of the compound of Example 3, the “Form A crystal,” was obtained from each of the above methods A through F.

¹H NMR (400 MHz, DMSO-d₆): 8.41 (d, J=1.6 Hz, 1H); 7.57 (m, 1H); 7.50 (d, J=8.0 Hz, 1H); 6.58 (t, J=5.8 Hz, 1H); 6.02 (d, J=1.6 Hz, 1H); 5.08 (d, J=8.0 Hz, 1H); 4.90 (m, 1H); 4.17 (t, J=8.6 Hz, 1H); 3.86 (m, 3H); 3.48 (t, J=5.6 Hz, 2H); 2.49 (overlapped with DMSO-d₆, 2H).

Elemental Analysis: Found (%) C, 52.91; H, 3.71; N, 13.71. Calcd C, 52.95; H, 3.70; N, 13.72.

HRMS: 431.0938 [M+Na].

DSC: sharp upward peak in the DSC chromatogram indicating a single endothermic event at about 166-168° C. See FIG. 1.

XRPD: major peaks at about 8.5-8.6 and 23.0-23.1° 2θ. SCAN: 3.0/45.0082/0.01971/17.4 (sec), Cu(40 kV, 40 mA), I(max)=72071. See FIG. 2.

IR (in KBr; cm⁻¹): 3403.4, 1744.2, 1665.7, 1594.0, and 1519.3. See FIG. 3.

UV (in MeOH): maximum absorption at 318 nm.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While the present invention has been described herein in conjunction with a preferred aspect, a person with ordinary skills in the art, after reading the foregoing specification, can affect changes, substitutions of equivalents and other types of alterations to methods and processes as set forth herein. Each aspect described above can also have included or incorporated therewith such variations or aspects as disclosed in regard to any or all of the other aspects. The present invention is also not to be limited in terms of the particular aspects described herein, which are intended as single illustrations of individual aspects of the invention. Many modifications and variations of this invention can be made without departing from its spirit and scope, as will be apparent to those skilled in the art. Functionally equivalent methods within the scope of this invention, in addition to those enumerated herein, will be apparent to those skilled in the art from the foregoing descriptions. It is to be understood that this invention is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

1. A process for the synthesis or manufacture of a compound of formula I: 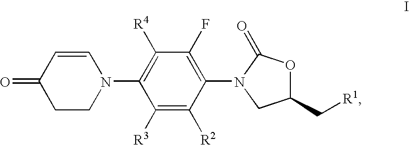
or a pharmaceutical acceptable crystal form, salt, hydrate, or solvate thereof;
the process comprising the following steps (a) to (c):
a) combining a silyl enol ether compound of formula III, O-alkyl-O′-allyl carbonate, a Pd(II) compound, and a fluorinated nitrobenzene compound in an aprotic solvent to form an N-aryl-4-(2,3-dihydro)pyridone compound of formula IV: 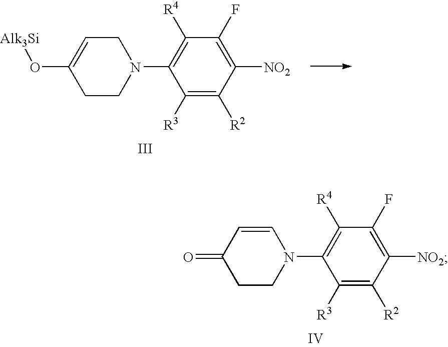
and, upon reduction of the compound of formula IV into a substituted aniline, and acylation of the resulting aniline into a carbamate compound of formula VI; further
b) combining the carbamate compound of formula VI, an epoxide compound, and a base in an aprotic solvent to form an oxazolidinone compound of formula VII: 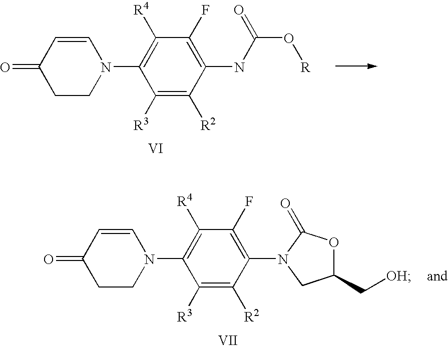
c) converting the compound of formula VII, optionally through one or more intermediates, into the compound of formula I, wherein R¹ is other than OH: 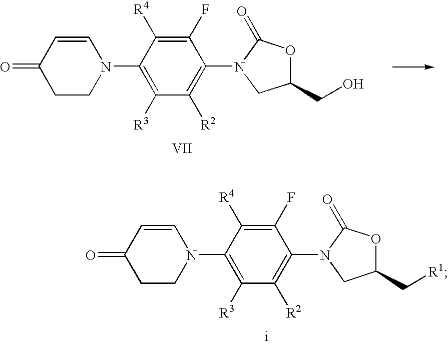
wherein
R is C₁₋₁₂alkyl, C₃₋₆cycloalkyl, or arylalkyl;
R¹ is NHC(═O)R⁵, OH, R⁵OH, NHC(═S)R⁵, NHC(═NCN)R⁵, NH-Het¹, O-Het¹, S-Het¹, or Het²; wherein R⁵ is H, NH₂, NHC₁₋₄alkyl, C₁ alkyl, C₃₋₆cycloalkyl, C₂₋₄alkenyl, C₂₋₄alkynyl, C₁₋₄heteroalkyl, Het¹, Het², (CH₂)ₘC(═O)C₁₋₄alkyl, OC₁₋₄alkyl, SC₁₋₄alkyl, (CH₂)ₘC₃₋₆cycloalkyl, (CH₂)ₘC(═O)-aryl, or (CH₂)ₘC(═O)—Het¹; m is 0, 1, or 2; Het¹ is independently a C-linked 5 or 6 membered heterocyclic ring having 1 to 4 heteroatoms selected from the group consisting of oxygen, nitrogen, and sulfur within the ring; and Het² is independently an N-linked 5 or 6 membered heterocyclic ring having 1 to 4 nitrogen atoms and optionally having one oxygen or sulfur within the ring;
R² is H or F; and
R³ and R⁴ are independently H, F, Cl, or CN.

2. The process according to claim 1 wherein R² is H, and R³ and R⁴ are both F.

3. The process according to claim 1 wherein at least one of the group consisting of R², R³, and R⁴ is F.

4. The process according to claim 1 wherein R¹ is OH.

5. The process according to claim 1 wherein R¹ is (5-R⁶-isoxazol-3-yl)oxy or (5-R⁶-isoxazol-3-yl)amino, and wherein R⁶ is H or C₁₋₆alkyl.

6. The process according to claim 1 wherein R¹ is (isoxazole-3-yl)amino.

7. The process according to claim 1 wherein R¹ is (isoxazole-3-yl)amino, R² is H, and R³ and R⁴ are both F.

8. The process according to claim 1 wherein R¹ is 4-R⁷-triazole-1-yl, and R⁷ is H, F, CN, or C₁₋₆alkyl.

9. The process according to claim 1 wherein the compound of formula I is selected from any of the following structures:

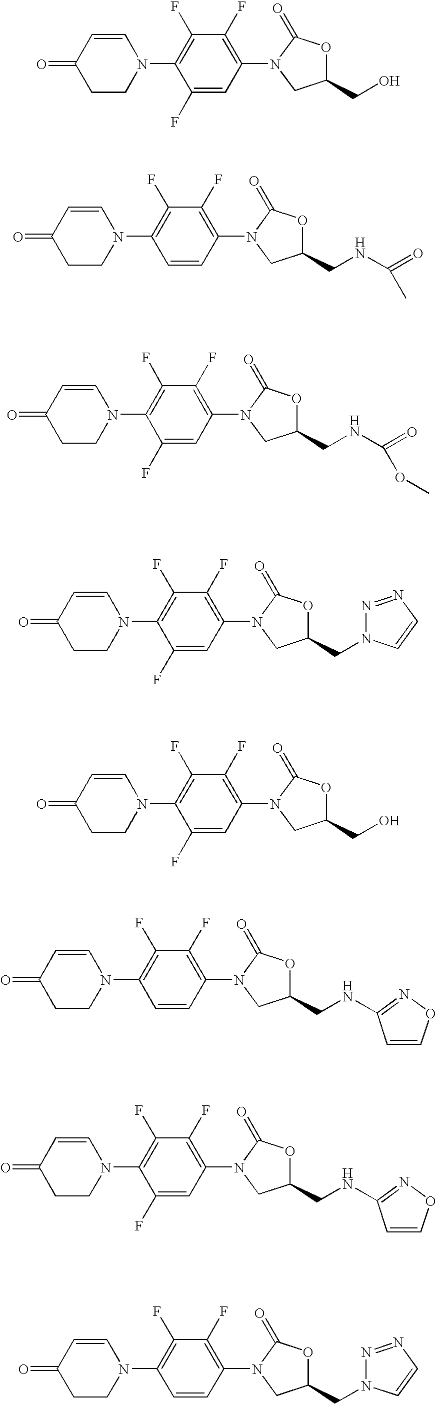

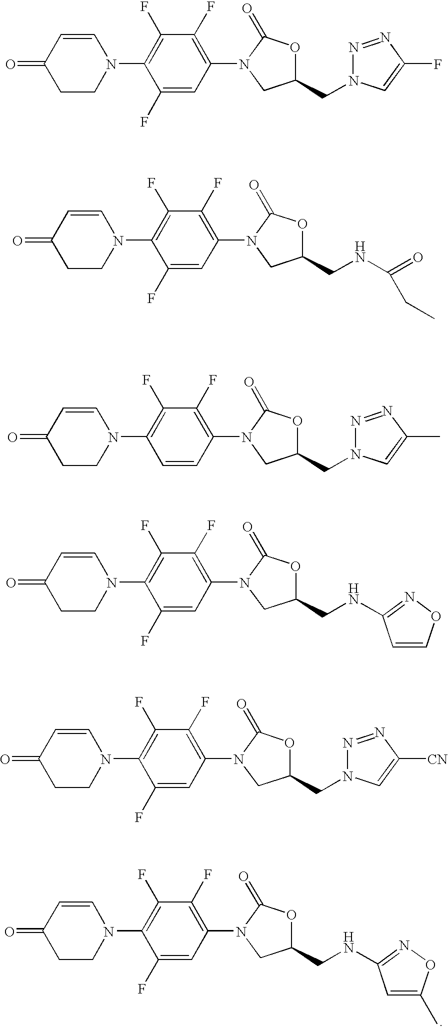

10. A process for the synthesis or manufacture of a compound of formula II comprising combining a 4-piperidone compound with a substituted 2-fluoronitrobenzene compound and a base in an aprotic solvent to form an N-aryl-4-piperidone compound of formula II: 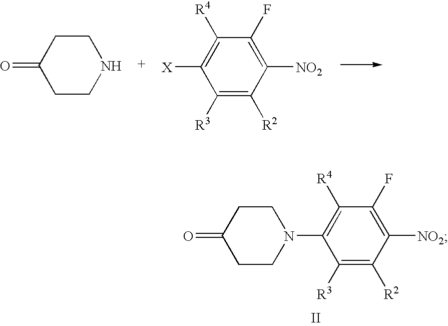
wherein
X is F, Cl, Br, I;
R² is H or F; and
R³ and R⁴ are independently H, F, Cl, or CN.

11. A process for the synthesis or manufacture of a compound of formula III comprising combining an N-aryl-4-piperidone compound of a compound of formula II with a trialkylsilyl compound Alk₃SiX (wherein X is halo, alkylsulfonate, or triflate) and a base in an aprotic solvent to form a silyl enol ether compound of formula III: 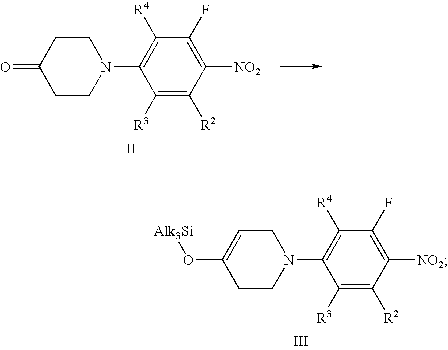
wherein
R² is H or F; and
R³ and R⁴ are independently H, F, Cl, or CN.

12. A process for the synthesis or manufacture of a compound of formula IV comprising combining a silyl enol ether compound of formula III, O-alkyl-O′-allyl carbonate, a Pd(II) compound, and an fluorinated nitrobenzene compound in an aprotic solvent to form an N-aryl-4-(2,3-dihydro)pyridone compound of formula IV: 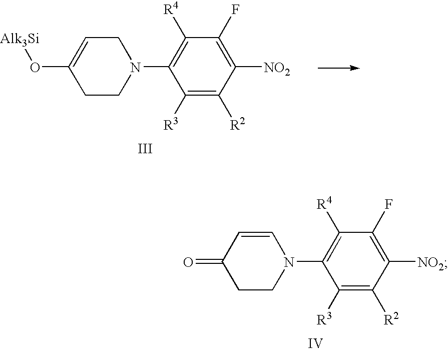
wherein
R² is H or F; and
R³ and R⁴ are independently H, F, Cl, or CN.

13. A process for the synthesis or manufacture of a compound of formula V comprising combining an N-aryl-4-(2,3-dihydro)pyridone compound of formula IV with a metal powder (selected from Fe, Sn, or Zn) in acidic aqueous solution, or combining a N-aryl-4-(2,3-dihydro)pyridone compound of formula IV with a hydrogen source and a Pd catalyst, to form an aniline compound of formula V: 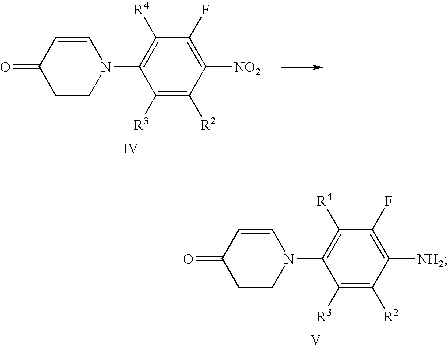
wherein
R² is H or F; and
R³ and R⁴ are independently H, F, Cl, or CN.

14. A process for the synthesis or manufacture of a compound of formula VI comprising combining an aniline compound of formula V, alkyl chloroformate, and a base in aprotic solvent to form a carbamate compound of formula VI: 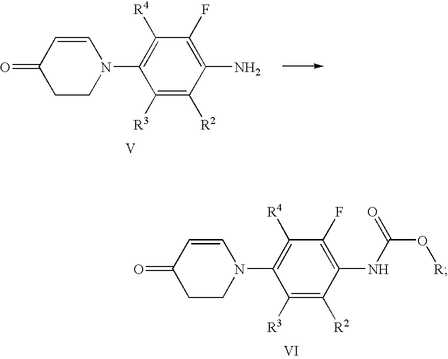
wherein
R is C₁₋₁₂alkyl, C₃₋₆cycloalkyl, or arylalkyl
R² is H or F; and
R³ and R⁴ are independently H, F, Cl, or CN.

15. A process for the synthesis or manufacture of a compound of formula VII comprising combining a carbamate compound of formula VI, an epoxide compound, and a base in an aprotic solvent to form an oxazolidinone compound of formula VII: 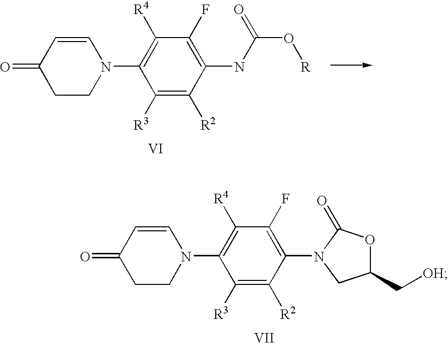
wherein
R is C₁₋₁₂alkyl, C₃₋₆cycloalkyl, aryl, or arylalkyl;
R² is H or F; and
R³ and R⁴ are independently H, F, Cl, or CN.

16. A process for the synthesis or manufacture of a compound of formula VIII comprising combining an oxazolidinone compound of formula VII and a compound R⁹SO₂Cl in an aprotic solvent and a base to form a sulfonate oxazolidinone compound of formula VIII: 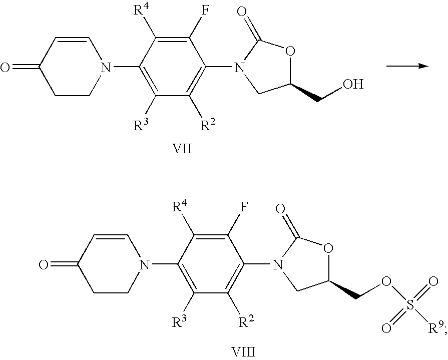
wherein
R² is H or F;
R³ and R⁴ are independently H, F, Cl, or CN; and
R⁹ is C₁₋₁₂alkyl, C₃₋₆cycloalkyl, aryl, or arylalkyl.

17. A process for the synthesis or manufacture of a compound of formula IX comprising combining a sulfonate compound of formula VIII, 3-(PG)NH-5-R⁶-isoxazole, and a base in an aprotic solvent to form a compound of formula IX: 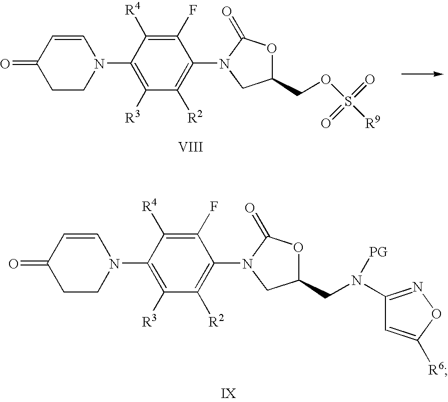
wherein
R² is H or F;
R³ and R⁴ are independently H, F, Cl, or CN;
R⁶ is H or C₁₋₆alkyl;
R⁹ is C₁₋₁₂alkyl, C₃₋₆cycloalkyl, aryl, or arylalkyl; and
PG is H or N-protective substituent selected from C₁₋₆alkoxycarbonyl, benzyloxycarbonyl, trichloroethoxycarbonyl, tert-butoxycarbonyl, para-methoxybenzyl, or dimethoxybenzyl.

18. A process for the synthesis or manufacture of a compound of formula I comprising combining a compound of formula IX, 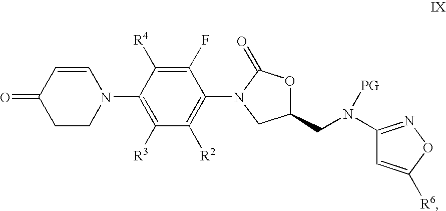
with an N-protection removing agent to form the following compound of formula I:

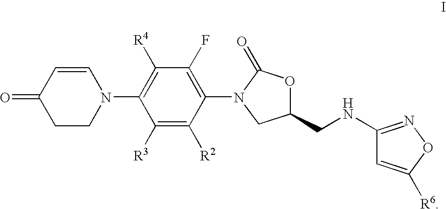

19. The process according to claim 10 wherein the substituted 2-fluoronitrobenzene is 2,3,4-trifluoronitrobenzene or 2,3,4,5-tetrafluoronitrobenzene; the aprotic solvent is N-methylpyrrolidin-2-one; the base is N,N-diisopropyl-N′-ethylamine; and the process is performed at temperatures between −20 and 60° C.

20. The process according to claim 11, wherein the Alk₃SiX reagent is TMSCl or TMSOTf; the aprotic solvent is tetrahydrofuran; the base is triethylamine; and the process is performed at temperatures between −10 and 50° C.

21. The process according to claim 1, wherein the O-alkyl-O′-allyl carbonate is O-methyl-O′-allyl carbonate; the Pd(II) compound is Pd(OAc)₂; the aprotic solvent is DMSO; and the process is performed at temperatures between 0 and 60° C.

22. The process according to claim 13, wherein the metal powder is Fe; the hydrogen source is H₂ gas, or an organic hydrogen source such as cyclohexene or a formic acid reagent; and the Pd catalyst is Pd/C, Pd/CaCO₃, Pd(OH)₂, or Pd/C/quinoline.

23. The process according to claim 14, wherein the alkyl chloroformate is isobutyl chloroformate or benzyl chloroformate; the aprotic solvent is DCM; the base is pyridine; and the process is performed at temperatures between −10 and 60° C.

24. The process according to claim 1 wherein the epoxide compound is (R)-glycidyl butyrate; the aprotic solvent is THF or MeCN, or a mixture of THF and MeCN in any ratio; the base is lithium or potassium t-butoxide; and the process is performed at temperatures between −20 and 60° C.

25. The process according to claim 24, wherein the base is lithium or potassium t-butoxide; and the process is performed at temperatures between −10 and 25° C.

26. The process according to claim 16, wherein R⁹SO₂Cl is CH₃SO₂Cl; and the base is triethylamine.

27. The process according to claim 17, wherein the substituted aminoheterocycle is 3-[N-(tert-butoxycarbonyl)amino]isoxazole; the aprotic solvent is DMF; and the base is potassium t-butoxide.

28. The process according to claim 17, wherein the base is potassium tert-butoxide.

29. The process according to claim 18, wherein the N-protection removing agent is 10-38% aqueous HCl or TMSCl.

30. The process according to claim 18, wherein the N-protection removing agent is 25-38% aqueous HCl in EtOH/EtOAc, wherein the HCl, EtOH and EtOAc are in any ratio.

31. The process according to claim 18, wherein the N-protection removing agent is 38% aqueous HCl in EtOH/EtOAc, wherein the HCl, EtOH and EtOAc are in any ratio between 1:1:1 to 3:1:3, respectively.

32. The process according to claim 21, wherein the fluorinated nitrobenzene compound is 2,3,4,5-tetrafluoronitrobenzene or 2,3,4-trifluoronitrobenzene, and wherein the amount of 2,3,4,5-tetrafluoronitrobenzene or 2,3,4-trifluoronitrobenzene is in the range of 5-70 molar %.

33. The process according to claim 21, wherein the fluorinated nitrobenzene compound is 2,3,4,5-tetrafluoronitrobenzene or 2,3,4-trifluoronitrobenzene, and wherein the amount of 2,3,4,5-tetrafluoronitrobenzene or 2,3,4-trifluoronitrobenzene is in the range of 40-60 molar %.

34. A crystalline form of the compound of formula:

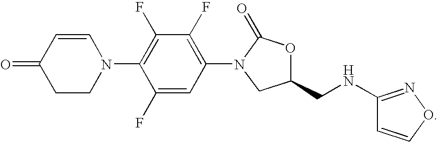

35. The crystalline form of claim 34, which exhibits in differential scanning calorimetry a single endothermic event at about 166-168° C.

36. The crystalline form of claim 35, which exhibits a melting point between about 166.9 and about 168.3° C.

38. The crystalline form of claim 35, which exhibits major X-ray powder diffraction peaks at about 8.5 to 8.6 and at about 23.0 to 23.1 °2θ using Cu Kα radiation.

39. The crystalline form of claim 35, which exhibits major infrared absorbance peaks at about 3403.4, about 1744.2, about 1665.7, about 1594.0, and about 1519.3 cm⁻¹.

40. The crystalline form of claim 35, which is obtained by crystallizing the compound of formula: 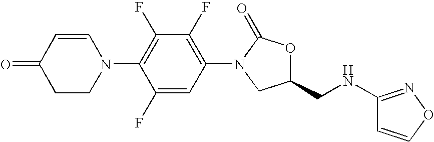
from a solvent selected from the group consisting of ethanol, ethyl acetate, hexane, petroleum ether, methyl t-butyl ether, water, and mixtures thereof.
